(19) United States
(12) Patent Application Publication
(10) Pub. No.: US 2025/0205260 A1
(43) Pub. Date: Jun. 26, 2025

(54) ADJUNCTIVE TREATMENT OF MYCOBACTERIAL DISEASES

(71) Applicants: UNIVERSITY OF SOUTHERN CALIFORNIA, Los Angeles, CA (US); CENTRAL MICHIGAN UNIVERSITY, Mount Pleasant, MI (US); UNIVERSITY OF MAINE SYSTEM BOARD OF TRUSTEES, Orono, ME (US)

(72) Inventors: Hyungjin EOH, Los Angeles, CA (US); Benjamin SWARTS, Mount Pleasant, MI (US); Peter WOODRUFF, Orono, ME (US); Jae Jin LEE, Los Angeles, CA (US)

(73) Assignees: UNIVERSITY OF SOUTHERN CALIFORNIA, Los Angeles, CA (US); CENTRAL MICHIGAN UNIVERSITY, Mount Pleasant, MI (US); UNIVERSITY OF MAINE SYSTEM BOARD OF TRUSTEES, Orono, ME (US)

(21) Appl. No.: 18/847,759

(22) PCT Filed: Mar. 21, 2023
(86) PCT No.: PCT/US2023/015729

§ 371 Date: Sep. 17, 2024

Related U.S. Application Data
(60) Provisional application No. 63/322,474, filed on Mar. 22, 2022.

(51) Int. Cl. A61K 31/7016; A61K 31/133; A61K 31/4409; A61K 31/47; A61K 31/4709; A61K 31/496; A61K 31/4965; A61K 31/606; A61K 31/7036; A61K 31/706; A61P 31/06

(52) U.S. Cl. CPC A61K 31/7016; A61K 31/133; A61K 31/4409; A61K 31/47; A61K 31/4709; A61K 31/496; A61K 31/4965; A61K 31/606; A61K 31/7036; A61K 31/706; A61P 31/06

(57) ABSTRACT

Disclosed herein are azidodeoxy- and aminodeoxy trehalose (TreAz and TreNH₂) compounds that inhibit Mycobacterium tuberculosis (an etiological agent of TB) biofilm (an in vitro model of intent TB infection) formation via blocking of the adaptive strategy used by M. tuberculosis form biofilm, termed trehalose catalytic shift. Prior to our work, such compounds were never tested for inhibition of mycobacterial biofilm formation. Our work defined this class of compounds' mechanism of action and showed for the first time that they sensitize drug-tolerant mycobacteria to existing clinically used antibiotics. The disclosed compounds are potential drugs for adjunctive treatment of TB and related mycobacterial diseases.

### RELATED APPLICATIONS

This application claims priority under 35 U.S.C. § 119 (e) to U.S. Provisional Patent Application No. 63/322,474 filed Mar. 22, 2022, which is incorporated herein by reference.

### GOVERNMENT SUPPORT

This invention was made with government support under Grant Nos. R15 AI117670, R21 AI139386, and R56 AI143870 awarded by the National Institutes of Health. The government has certain rights in the invention.

### BACKGROUND OF THE INVENTION

Tuberculosis (TB) is a global pandemic that impacts low-income counties. The World Health Organization (WHO) reported that 10.5 million people worldwide were diagnosed with TB each year and 1.5 million people died from the disease. TB is curable with current antibiotic regimens, but outcomes have been unsuccessful largely because of latent TB infection and the emergence of drug resistant mutants. As a chemotherapeutic method for drug resistant TB patients, the method has a very low rate of success. New TB antibiotic development has thus reemerged as a major area of unmet medical need.

Previous research on M. tuberculosis trehalose catalytic shift are summarized by the following points: In vitro biofilm culture enriched with M. tuberculosis in a latent state. M. tuberculosis biofilm was used to model M. tuberculosis in a latent infection state. Untargeted metabolomics of M. tuberculosis cultured in a biofilm revealed that trehalose metabolism was significantly altered during the latent state. A shift in trehalose carbon flux, a strategy termed trehalose catalytic shift, is associated with increased biosynthesis of central carbon metabolism intermediates to sustain energy and antioxidants required for M. tuberculosis latent viability. TreS (trehalose synthase)-deficient M. tuberculosis mutant (ΔtreS) failed to enter a latent state, thereby being hypersensitive to conventional TB antibiotics. In a TB mouse model, ΔtreS showed growth comparable with wild type during the initial phase, followed by a latent stage defect in bacterial load. The TreS requirement for persistent infection with M. tuberculosis reflected either a need for the production of additional trehalose or for mobilization of stored trehalose into maltose. TreS and the trehalose catalytic shift are a potential source of adjunctive therapeutic targets to synergize the antibiotic effects of existing TB antibiotic regimens by preventing M. tuberculosis from entering a drug-tolerant latent state.

Previous research on aminodeoxy trehalose analogues are summarized by the following points: 2-amino-2-deoxy-D-trehalose (2-TreNH₂) exhibited inhibition of M. tuberculosis planktonic growth. 3-Amino-3-deoxy-D-trehalose (3-TreNH₂), isolated from Nocardiopsis trehalosei, was not tested against Mycobacterium species. 4-Amino-4-deoxy-D-trehalose (4-TreNH₂), isolated from Streptomyces, did not exhibit inhibition of M. smegmatis planktonic growth. This compound was not tested in M. tuberculosis. 6-Trehalosamine (6-TreNH₂) did not exhibit inhibition of M. tuberculosis planktonic growth. None of these compounds were tested for biofilm inhibition and a mechanism of action was never defined.

Previous research on azidodeoxy trehalose analogues are summarized by the following points: 2-azido-2-deoxy-D-trehalose (2-TreAz) exhibited planktonic growth inhibition of M. smegmatis. 3-Azido-3-deoxy-D-trehalose (3-TreAz) did not exhibit inhibition of M. smegmatis planktonic growth. 4-Azido-4-deoxy-D-trehalose (4-TreAz) did not exhibit inhibition of M. smegmatis planktonic growth. 6-Azido-6-deoxy-D-trehalose (6-TreAz) was shown to inhibit growth of M. aurum, M. tuberculosis, and M. smegmatis. None of the compounds were tested for biofilm inhibition and mechanism of action was never defined.

The problem is the emergence of drug resistant mutants of TB. Accordingly, there is a need for alternative treatments for TB.

### SUMMARY

Chemical synthesis of trehalose compounds and methods of therapeutic interventions against both active and latent tuberculosis infection are disclosed.

Accordingly, this disclosure provides a method for treating a mycobacterial disease in a subject in need thereof comprising administering to the subject a therapeutically effective amount of a mycobacterial antibiotic and a therapeutically effective amount of one or more deoxy trehaloses that are capable of sensitizing mycobacteria to the mycobacterial antibiotic, wherein the mycobacterial disease in the subject is thereby treated.

Also, this disclosure provides a method for inhibiting mycobacterial growth, mycobacterial planktonic growth, or mycobacterial biofilm formation comprising contacting a mycobacteria and an effective amount of one or more deoxy trehaloses, wherein mycobacterial growth, mycobacterial planktonic growth, or mycobacterial biofilm formation is thereby inhibited.

Additionally, this disclosure provides a composition comprising a mycobacterial antibiotic and one or more deoxy trehaloses of Formula I:

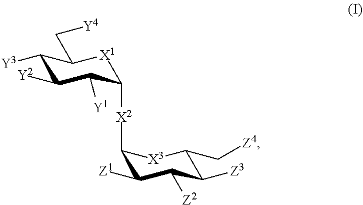

or an epimer thereof;
wherein
   X¹, X², and X³ are each independently O or NR¹;
   Y¹, Y², Y³ and Y⁴ are each independently OR², N₃, or N(R¹)₂;
   Z¹, Z², Z³ and Z⁴ are each independently OR², N₃, or N(R¹)₂; and
   R¹ and R² are each independently H or (C₁-C₆)alkyl;
wherein each of the one or more deoxy trehaloses of Formula I comprises at least one N₃, or N(R¹)₂ moiety.

In some embodiments of this technology the one or more deoxy trehaloses are (2R,3R,4S,5S,6R)-2-(((2R,3R,4R,5S,6R)-3-amino-4,5-dihydroxy-6-(hydroxymethyl)tetrahydro-2H-pyran-2-yl)oxy)-6-(hydroxymethyl)tetrahydro-2H-pyran-3,4,5-triol (2-TreNH₂), (2R,3R,4S,5S,6R)-2-(((2R,3R,4S,5S,6S)-5-amino-3,4-dihydroxy-6-(hydroxymethyl)tetrahydro-2H-pyran-2-yl)oxy)-6-(hydroxymethyl)tetrahydro-2H-pyran-3,4,5-triol (4-TreNH₂), or (2R,3S,4S,5R,6R)-2-(aminomethyl)-6-(((2R,3R,4S,5S,6R)-3,4,5-trihydroxy-6-(hydroxymethyl)tetrahydro-2H-pyran-2-yl)oxy)tetrahydro-2H-pyran-3,4,5-triol (6-TreNH₂).

The invention provides novel compounds of Formula I, II, or III (e.g., a compound of Formula III wherein the compound is not 2-, 3- or 4-TreNH₂), intermediates for the synthesis of compounds of Formulas I-III, as well as methods of preparing compounds of Formulas I-III. The invention also provides compounds of Formulas I-III that are useful as intermediates for the synthesis of other useful compounds. The invention provides for the use of compounds of Formulas I-III for the manufacture of medicaments useful for the treatment of bacterial infections in a mammal, such as a human.

The invention provides for the use of the compositions described herein for use in medical therapy. The medical therapy can treat bacterial infections, for example, tuberculosis. The invention also provides for the use of a composition as described herein for the manufacture of a medicament to treat a disease in a mammal, for example, tuberculosis in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-D. Anti-M. tuberculosis biofilm activity of 6-treAz (6-azido-6-deoxy-D-trehalose). The effects of various concentrations of 6-treAz treatment on M. tuberculosis biofilms were monitored by crystal violet (CV) staining assay (A and B). No discernible effect of 6-treAz on M. tuberculosis planktonic growth was detected (C). The effect of 6-treAz on TreS mediated maltose production. Y-axis is the amount of maltose produced (D).

FIG. 2A-D. Metabolic impact of various concentrations of 6-treAz on M. tuberculosis biofilm metabolism.

FIG. 3. Antimycobacterial synergy of 6-treAz with known TB antibiotics, BDQ (bedaquiline). All values are the average of biological triplicates±s.e.m. ***, P<0.001 by ANOVA.

FIG. 4. M. tuberculosis metabolome altered after treatment with either first-line TB drug (INH) or second-line TB drug (BDQ) was clearly different from that of ΔtreS but became similar by co-treatment with 6-treAz.

FIG. 5A-D. Evaluation of 2-TreAz and 2-TreNH₂ against M. smegmatis (A) planktonic growth and (B) biofilm formation and M. tuberculosis (C) planktonic growth and (D) biofilm formation. INH, isoniazid; NT, not treated.

FIG. 6A-D. Evaluation of 3-TreAz and 3-TreNH₂ against M. smegmatis (A) planktonic growth and (B) biofilm formation and M. tuberculosis (C) planktonic growth and (D) biofilm formation. INH, isoniazid; NT, not treated.

FIG. 7A-D. Evaluation of 4-TreAz and 4-TreNH₂ against M. smegmatis (A) planktonic growth and (B) biofilm formation and M. tuberculosis (C) planktonic growth and (D) biofilm formation. INH, isoniazid; NT, not treated.

FIG. 8A-D. Evaluation of 6-TreAz and 6-TreNH₂ against M. smegmatis (A) planktonic growth and (B) biofilm formation and M. tuberculosis (C) planktonic growth and (D) biofilm formation. INH, isoniazid; NT, not treated.

FIG. 9A-C. M. tuberculosis metabolomics analysis after treatment with various TreNH₂. (A) Clustered heatmap depicting ˜410 metabolites of M. tuberculosis after treatment with 2, 4, and 6-TreNH₂. The profiles were compared with that of ΔtreS. (B) Principal component analysis (PCA) using M. tuberculosis metabolome. Three dimensional PCA score plots reveal that metabolome profiles of 2- and 4-TreNH₂ are similar to those of ΔtreS but different from those of None or 6-TreNH₂. (C) Molecular phylogeny generated by M. tuberculosis metabolome profiles.

FIG. 10. Drug resistance phenotypes (MIC shift assay) for H37Rv lab strain in the presence of varying doses of anti-TB antibiotics such as isoniazid (INH), rifampicin (RIF), or bedaquiline (BDQ) with/without trehalosamine compounds (2-, 4-, or 6-TreNH₂). MIC values after combination treatment with antibiotic and trehalosamine compound were calculated by monitoring OD₅₉₅ relative to that of no treatment control. Data represent mean #s.e.m. for technical triplicates. Results are representative data from at least two independent experiments. 2-TreNH₂ (triangles) showed antimicrobial synergy against H37Rv level similar to that of ΔtreS (squares).

FIG. 11A-B. Epi-2-TreAz selectively inhibits biofilm formation of M. smegmatis. Epi-2-TreNH₂, epi-4-TreAz, and epi-4-TreNH₂ treatment showed no inhibitory effects on M. smegmatis biofilm formation.

FIG. 12A-B. Epi-2-TreAz completely inhibits planktonic growth of M. tuberculosis but epi-2-TreNH₂, epi-4-TreAz, and epi-4-TreNH₂ have no inhibitory effects on M. tuberculosis planktonic growth (A). Epi-2-TreAz also completely inhibits biofilm formation of M. tuberculosis. However, Epi-2-TreNH₂, epi-4-TreAz, and epi-4-TreNH₂ do not (B).

### DETAILED DESCRIPTION

We disclose that azidodeoxy- and aminodeoxy trehalose (TreAz and TreNH₂) compounds that inhibit Mycobacterium tuberculosis (an etiological agent of TB) biofilm (an in vitro model of intent TB infection) formation via blocking the adaptive strategy used by M. tuberculosis form biofilm, termed trehalose catalytic shift. Prior to our work, such compounds were never tested for inhibition of mycobacterial biofilm formation. In particular, 4-TreNH₂ was tested for planktonic growth in M. smegmatis and no activity was observed, while M. tuberculosis was not even tested. Therefore, our finding that 4-TreNH₂ inhibits M. tuberculosis planktonic growth and biofilm formation is a new innovation in this field. Our work defines this class of compounds' mechanism of action and shows for the first time that they sensitize drug-tolerant mycobacteria to existing clinically used antibiotics. The disclosed compounds are potential drugs for adjunctive treatment of TB and related mycobacterial diseases.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability, necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, a patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

The term “heteroatom” refers to any atom in the periodic table that is not carbon or hydrogen. Typically, a heteroatom is O, S, N, P. The heteroatom may also be a halogen, metal or metalloid.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., halo, alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The terms “racemic mixture” and “racemate” refer to an equimolar mixture of two enantiomeric species, devoid of optical activity.

The term “IC₅₀” is generally defined as the concentration required to inhibit a specific biological or biochemical function by half, or to kill 50% of the cells in a designated time period, typically 24 hours.

The term “trehalose-catalytic shift” refers to an essential component of adaptive metabolism that not only permits survival in response to a dysregulated electron transport chain and ATP depletion, but also facilitates—either directly or indirectly—the accumulation of drug-resistant mutations in Mycobacterium tuberculosis. A catalytic shift of trehalose metabolism keeps trehalose and maltose away from the biosynthesis of cell wall trehalose monomycolate and trehalose dimycolate and channels trehalose and maltose into the biosynthesis of central carbon metabolism intermediates in order to maintain ATP and antioxidant biosynthetic activities.

### Embodiments of the Technology

This disclosure provides a method for treating a mycobacterial disease in a subject in need thereof comprising administering to a subject a therapeutically effective amount of a mycobacterial antibiotic and a therapeutically effective amount of one or more deoxy trehaloses that are capable of sensitizing mycobacteria to the mycobacterial antibiotic, wherein the mycobacterial disease in the subject is thereby treated.

In various embodiments, the sensitization results from inhibition or disruption of mycobacterial biofilm formation. In various embodiments, the one or more deoxy trehaloses comprise an aminodeoxy trehalose, an azidodeoxy trehalose, or a combination thereof.

In various embodiments, the one or more deoxy trehaloses is an aminodeoxy trehalose, and the aminodeoxy trehalose is:

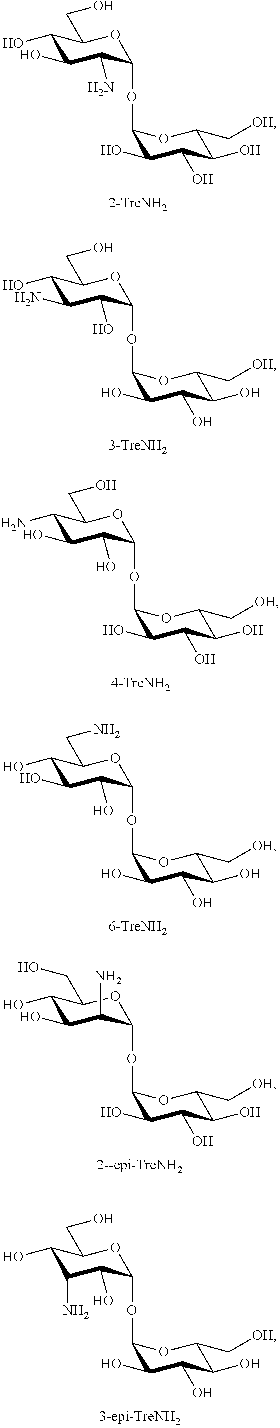

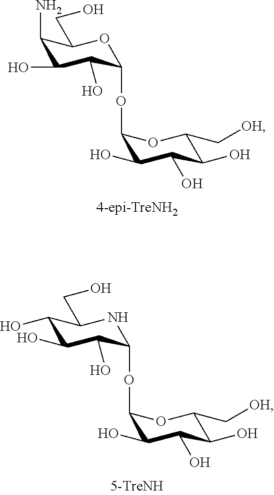

or a combination thereof.

In various embodiments, the aminodeoxy trehalose is: (2R,3R,4S,5S,6R)-2-(((2R,3R,4R,5S,6R)-3-amino-4,5-dihydroxy-6-(hydroxymethyl)tetrahydro-2H-pyran-2-yl)oxy)-6-(hydroxymethyl)tetrahydro-2H-pyran-3,4,5-triol (2-TreNH₂), (2R,3R,4S,5S,6R)-2-(((2R,3R,4S,5S,6S)-5-amino-3,4-dihydroxy-6-(hydroxymethyl)tetrahydro-2H-pyran-2-yl)oxy)-6-(hydroxymethyl)tetrahydro-2H-pyran-3,4,5-triol (4-TreNH₂), or (2R,3S,4S,5R,6R)-2-(aminomethyl)-6-(((2R,3R,4S,5S,6R)-3,4,5-trihydroxy-6-(hydroxymethyl)tetrahydro-2H-pyran-2-yl)oxy)tetrahydro-2H-pyran-3,4,5-triol (6-TreNH₂).

In various embodiments, the one or more deoxy trehaloses is an azidodeoxy trehalose, and the azidodeoxy trehalose is:

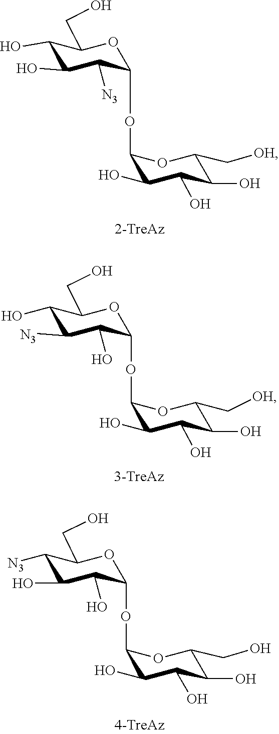

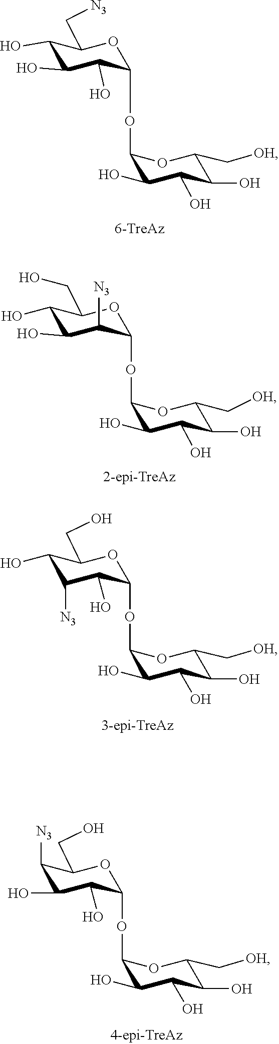

or a combination thereof.

In various embodiments, the mycobacterial antibiotic is bedaquiline, ethambutol, isoniazid, pyrazinamide, rifampicin, streptomycin, ciprofloxacin, moxifloxacin, p-aminosalicylic acid, or a combination thereof. In various embodiments, the mycobacterial antibiotic is bedaquiline or isoniazid.

In various embodiments, the mycobacterial disease is tuberculosis or leprosy. In various embodiments, the mycobacteria is Mycobacterium tuberculosis, Mycobacterium leprae, Mycobacterium smegmatis, Mycobacterium aurum, Mycobacterium avium, Mycobacterium abscessus, or Mycobacterium fortuitum. In various embodiments, the mycobacteria is Mycobacterium tuberculosis.

In various embodiments, the therapeutically effective amount of the one or more deoxy trehaloses is a systemic concentration of about 1 micromolar to about 1000 micromolar at steady state in the subject. In various embodiments, the therapeutically effective amount of the mycobacterial antibiotic is a systemic concentration of about 1 nanomolar to about 1000 micromolar at steady state in the subject.

In various embodiments, the therapeutically effective amount at steady state in the subject of the one or more deoxy trehaloses and/or the mycobacterial antibiotic is a systemic concentration of about 1 nanomolar (nM) to about 10 nM, about 10 nM to about 100 nM, about 100 nM to about 500 nM, about 500 nM to about 1 micromolar (mM), about 1 mM to about 10 mM, about 10 mM to about 50 mM, about 50 mM to about 100 mM, about 100 mM to about 200 mM, about 200 mM to about 300 mM, about 300 mM to about 400 mM, about 400 mM to about 500 mM, about 500 mM to about 600 mM, about 600 mM to about 700 mM, about 700 mM to about 800 mM, about 800 mM to about 900 mM, about 900 mM to about 1000 mM, about 1000 mM to about 1500 mM, or about 1500 mM to about 2000 mM.

This disclosure also provides a method for inhibiting mycobacterial growth, mycobacterial planktonic growth, or mycobacterial biofilm formation comprising contacting a mycobacteria and an effective amount of one or more deoxy trehaloses, wherein mycobacterial growth, mycobacterial planktonic growth, or mycobacterial biofilm formation is thereby inhibited.

In various embodiments, the method further comprises contacting the mycobacteria and an effective amount of a mycobacterial antibiotic, wherein the mycobacterial antibiotic and the one or more deoxy trehaloses are contacting the mycobacteria in combination.

In various embodiments, the mycobacterial antibiotic is bedaquiline, ethambutol, isoniazid, pyrazinamide, rifampicin, streptomycin, ciprofloxacin, moxifloxacin, p-aminosalicylic acid, or a combination thereof. In various embodiments, the mycobacteria is Mycobacterium tuberculosis, Mycobacterium leprae, Mycobacterium smegmatis, Mycobacterium aurum, Mycobacterium avium, Mycobacterium abscessus, or Mycobacterium fortuitum.

In various embodiments, inhibition of mycobacterial growth, mycobacterium planktonic growth, or mycobacterial biofilm formation occurs by inhibition of a trehalose-catalytic shift in a biosynthetic pathway of the mycobacteria.

In various embodiments, an effective amount of one or more deoxy trehaloses and/or an effective amount of a mycobacterial antibiotic is at a concentration of about 1 nanomolar to about 1000 micromolar. In various embodiments, the one or more deoxy trehaloses comprise an aminodeoxy trehalose, an azidodeoxy trehalose, or a combination thereof.

In various embodiments, the one or more deoxy trehaloses is: (2R,3R,4S,5S,6R)-2-(((2R,3R,4R,5S,6R)-3-amino-4,5-dihydroxy-6-(hydroxymethyl)tetrahydro-2H-pyran-2-yl)oxy)-6-(hydroxymethyl)tetrahydro-2H-pyran-3,4,5-triol (2-TreNH₂), (2R,3R,4S,5S,6R)-2-(((2R,3R,4S,5S,6R)-4-amino-3,5-dihydroxy-6-(hydroxymethyl)tetrahydro-2H-pyran-2-yl)oxy)-6-(hydroxymethyl)tetrahydro-2H-pyran-3,4,5-triol (3-TreNH₂), (2R,3R,4S,5S,6R)-2-(((2R,3R,4S,5S,6S)-5-amino-3,4-dihydroxy-6-(hydroxymethyl)tetrahydro-2H-pyran-2-yl)oxy)-6-(hydroxymethyl)tetrahydro-2H-pyran-3,4,5-triol (4-TreNH₂), (2R,3S,4S,5R,6R)-2-(aminomethyl)-6-(((2R,3R,4S,5S,6R)-3,4,5-trihydroxy-6-(hydroxymethyl)tetrahydro-2H-pyran-2-yl)oxy)tetrahydro-2H-pyran-3,4,5-triol (6-TreNH₂), (2R,3R,4S,5S,6R)-2-(((2R,3S,4R,5S,6R)-3-amino-4,5-dihydroxy-6-(hydroxymethyl)tetrahydro-2H-pyran-2-yl)oxy)-6-(hydroxymethyl)tetrahydro-2H-pyran-3,4,5-triol (2-epi-TreNH₂), (2R,3R,4S,5S,6R)-2-(((2R,3R,4R,5S,6R)-4-amino-3,5-dihydroxy-6-(hydroxymethyl)tetrahydro-2H-pyran-2-yl)oxy)-6-(hydroxymethyl)tetrahydro-2H-pyran-3,4,5-triol (3-epi-TreNH₂), (2R,3R,4S,5S,6R)-2-(((2R,3R,4S,5R,6S)-5-amino-3,4-dihydroxy-6-(hydroxymethyl)tetrahydro-2H-pyran-2-yl)oxy)-6-(hydroxymethyl)tetrahydro-2H-pyran-3,4,5-triol (4-epi-TreNH₂), (2R,3R,4S,5R,6R)-2-(hydroxymethyl)-6-(((2R,3R,4S,5S,6R)-3,4,5-trihydroxy-6-(hydroxymethyl)tetrahydro-2H-pyran-2-yl)oxy) piperidine-3,4,5-triol (5-TreNH), (2R,3R,4S,5S,6R)-2-(((2R,3R,4R,5S,6R)-3-azido-4,5-dihydroxy-6-(hydroxymethyl)tetrahydro-2H-pyran-2-yl)oxy)-6-(hydroxymethyl)tetrahydro-2H-pyran-3,4,5-triol (2-TreAz), (2R,3R,4S,5S,6R)-2-(((2R,3R,4S,5S,6R)-4-azido-3,5-dihydroxy-6-(hydroxymethyl)tetrahydro-2H-pyran-2-yl)oxy)-6-(hydroxymethyl)tetrahydro-2H-pyran-3,4,5-triol (3-TreAz), (2R,3R,4S,5S,6R)-2-(((2R,3R,4S,5S,6S)-5-azido-3,4-dihydroxy-6-(hydroxymethyl)tetrahydro-2H-pyran-2-yl)oxy)-6-(hydroxymethyl)tetrahydro-2H-pyran-3,4,5-triol (4-TreAz), (2R,3S,4S,5R,6R)-2-(azidomethyl)-6-(((2R,3R,4S,5S,6R)-3,4,5-trihydroxy-6-(hydroxymethyl)tetrahydro-2H-pyran-2-yl)oxy)tetrahydro-2H-pyran-3,4,5-triol (6-TreAz), (2R,3R,4S,5S,6R)-2-(((2R,3S,4R,5S,6R)-3-azido-4,5-dihydroxy-6-(hydroxymethyl)tetrahydro-2H-pyran-2-yl)oxy)-6-(hydroxymethyl)tetrahydro-2H-pyran-3,4,5-triol (2-epi-TreAz), (2R,3R,4S,5S,6R)-2-(((2R,3R,4R,5S,6R)-4-azido-3,5-dihydroxy-6-(hydroxymethyl)tetrahydro-2H-pyran-2-yl)oxy)-6-(hydroxymethyl)tetrahydro-2H-pyran-3,4,5-triol (3-epi-TreAz), (2R,3R,4S,5S,6R)-2-(((2R,3R,4S,5R,6S)-5-azido-3,4-dihydroxy-6-(hydroxymethyl)tetrahydro-2H-pyran-2-yl)oxy)-6-(hydroxymethyl)tetrahydro-2H-pyran-3,4,5-triol (4-epi-TreAz), or a combination thereof.

Additionally, this disclosure provides a deoxy trehalose of Formula I, II, or III:

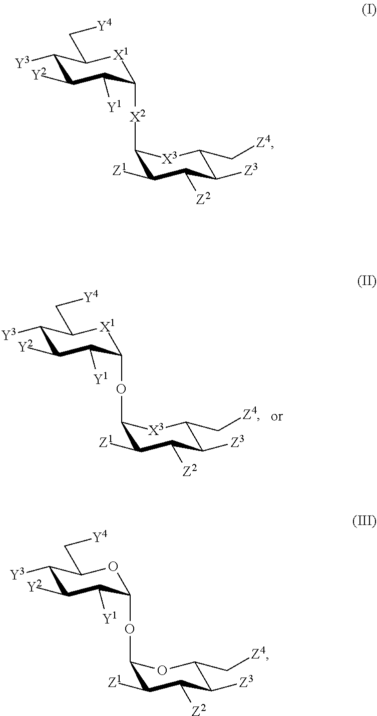

or an epimer thereof; wherein
X¹, X², and X³ are each independently O or NR¹;
Y¹, Y², Y³ and Y⁴ are each independently OR², N₃, or N(R¹)₂;
Z¹, Z², Z³ and Z⁴ are each independently OR², N₃, or N(R¹)₂; and
R¹ and R² are each independently H or alkyl, for example, (C₁-C₂₂)alkyl, (C₁-C₆)alkyl, (C₈-C₁₈)alkyl, or (C₁₀-C₂₂)alkyl;
   wherein the deoxy trehalose of Formula I comprises at least one N₃, or N(R¹)₂ moiety.

In some embodiments, R¹ is H. In some embodiments, R² is H. In some embodiments, R¹ and R² are H. In various embodiments, at least one of X¹, X², and X³ is O or NR¹. In various embodiments, at least one of Y¹, Y², Y³ and Y⁴ is N(R¹)₂ or N₃, or an epimer thereof. In various embodiments, at least one of Z¹, Z², Z³ and Z⁴ is N(R¹)₂ or N₃, or an epimer thereof. In various embodiments, Y¹, Y², Y³, Y⁴, Z¹, Z², Z³, and Z⁴ are all OR² except one of Y¹, Y², Y³, Y⁴, Z¹, Z², Z³, and Z⁴. is N(R¹)₂ or N₃.

In some embodiments, the deoxy trehalose of Formulas I-III is the 2-position epimer, the 3-position epimer, the 4-position epimer, the 2′-position epimer, the 3′-position epimer, or the 4′-position epimer. In various embodiments, Y¹ is not NH₂, Y² is not NH₂, Y³ is not NH₂, Y⁴ is not NH₂, or each of Y¹, Y², Y³ and Y⁴ are not NH₂. In further embodiments, one, two, or three of Y¹, Y², Y³, Y₄, Z¹, Z², Z³, and Z⁴ are not NH₂. In various embodiments, Y¹ is not N₃, Y² is not N₃, Y³ is not N₃, Y⁴ is not N₃, or each of Y¹, Y², Y³ and Y⁴ are not N₃. In further embodiments, one, two, or three of Y¹, Y², Y³, Y⁴, Z¹, Z², Z³, and Z⁴ are not N₃. In various embodiments, one, two, or three of Y¹, Y², Y³, Y₄, Z¹, Z², Z³, and Z⁴ are not OH.

Any one or more of the deoxy trehaloses of Formulas I-III can be used in a composition or method described herein. In various embodiments, the deoxy trehalose is: 2-TreNH₂, 3-TreNH₂, 4-TreNH₂, 6-TreNH₂, 2-epi-TreNH₂, 3-epi-TreNH₂, 4-epi-TreNH₂, 5-TreNH, 2-TreAz, 3-TreAz, 4-TreAz, 6-TreAz, 2-epi-TreAz, 3-epi-TreAz, or 4-epi-TreAz, or a combination thereof. In some embodiments, the deoxy trehalose is not one or more of the aminodeoxy trehaloses described herein or is not one or more of the azidodeoxy trehaloses described herein.

The disclosure further provides a composition comprising a deoxy trehalose of Formulas I, II, or III and a pharmaceutically acceptable excipient, optionally in combination with a mycobacterial antibiotic. In various embodiments, the mycobacterial antibiotic is present in the composition and the antibiotic is bedaquiline, ethambutol, isoniazid, pyrazinamide, rifampicin, streptomycin, ciprofloxacin, moxifloxacin, p-aminosalicylic acid, or a combination thereof.

### Results and Discussion

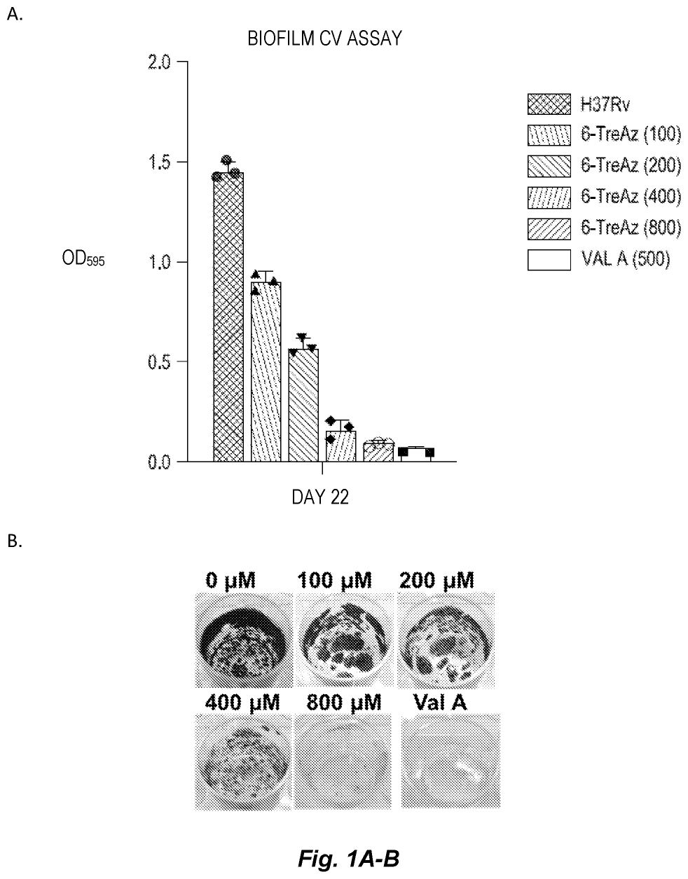

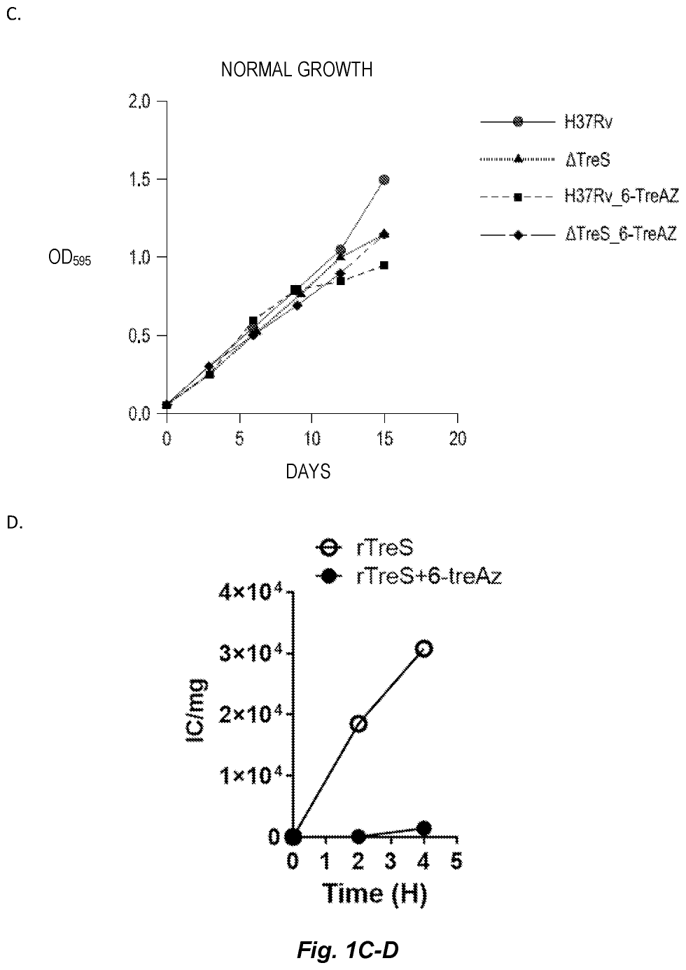

We discovered that trehalose analogues inhibited the formation of mycobacterial biofilms (an in vitro culture model used to recapitulate latent M. tuberculosis state) that are associated with drug-tolerant “persister” bacteria during infection. Certain trehalose analogues were known to inhibit M. smegmatis biofilm formation, including 6-treAz. Subsequently, a study showed the biofilm inhibition activity of 6-treAz also occurred in M. tuberculosis. This study provided the first in-depth mechanistic analysis for how trehalose-based inhibitors work in M. tuberculosis, showing that 6-TreAz blocked the TreS-mediated trehalose catalytic shift. Based upon the structural similarity between 6-treAz and a TreS substrate (trehalose), we showed a selective inhibitory activity against M. tuberculosis biofilm formation in a 6-treAz concentration dependent manner with no discernible effects on planktonic growth (FIG. 1A-C). We also examined whether anti-mycobacterial biofilm activity of 6-treAz is attributed to its competitive inhibition of the TreS and accompanied trehalose catalytic shift activity (FIG. 1D).

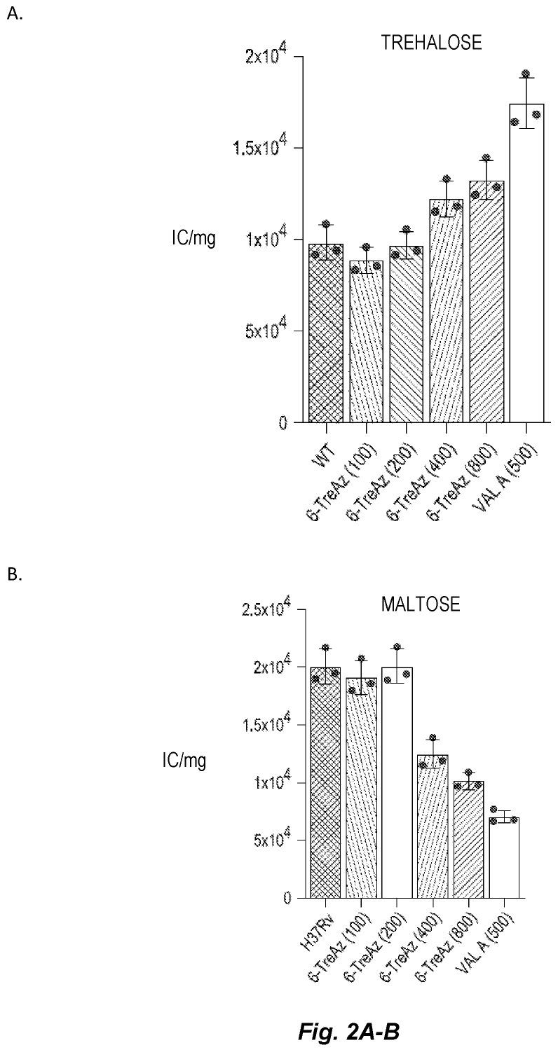

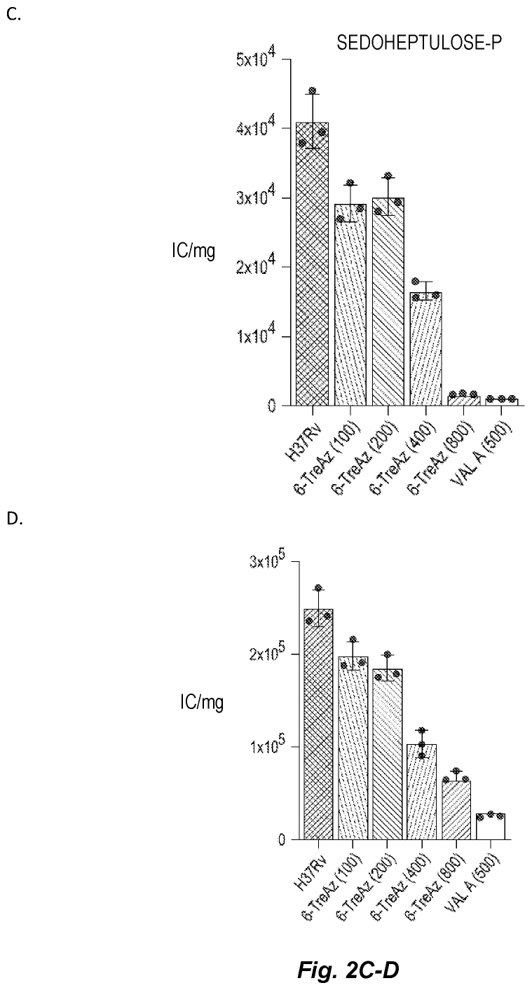

Treatment with 100 μM 6-treAz abolished maltose production in an in vitro TreS enzyme reaction. Consequently, treatment with at least 100 M 6-treAz in a WT biofilm culture significantly interfered with M. tuberculosis biofilm formation. 6-treAz-mediated inhibition of M. tuberculosis biofilm was accompanied by a dose-dependent inhibition of the trehalose catalytic shift, evidenced by gradual accumulation of trehalose with reciprocal depletion of maltose, glucose-P and sedoheptulose-P (Scheme1, FIG. 2).

Scheme 1. Intrabacterial pool sizes of M. tuberculosis biofilm intermediates in trehalose metabolism (trehalose and maltose), glycolysis (glucose-P), and pentose phosphate pathway (sedoheptulose-P). Validamyin A (Val A), a TreS-specific inhibitor, was used as a positive control.

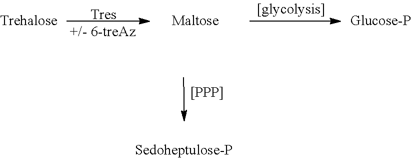

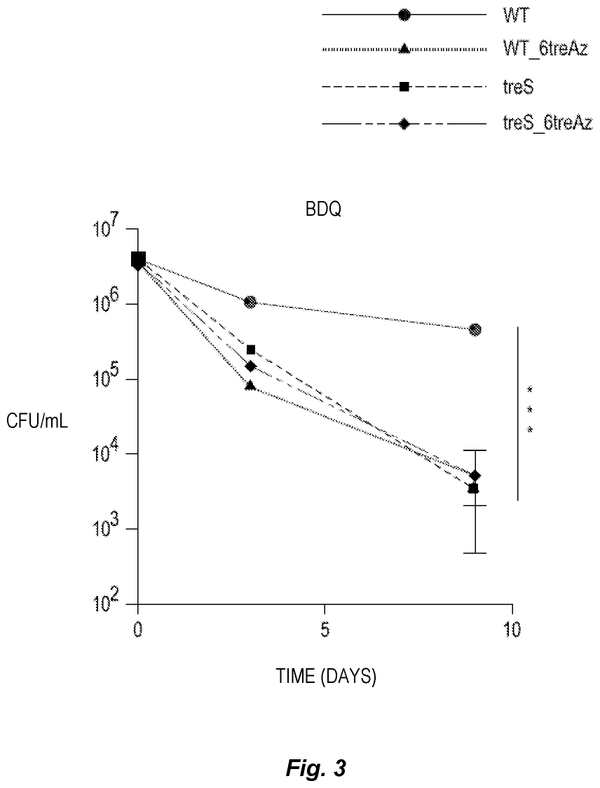

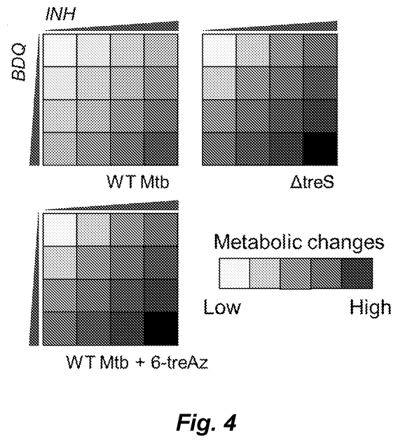

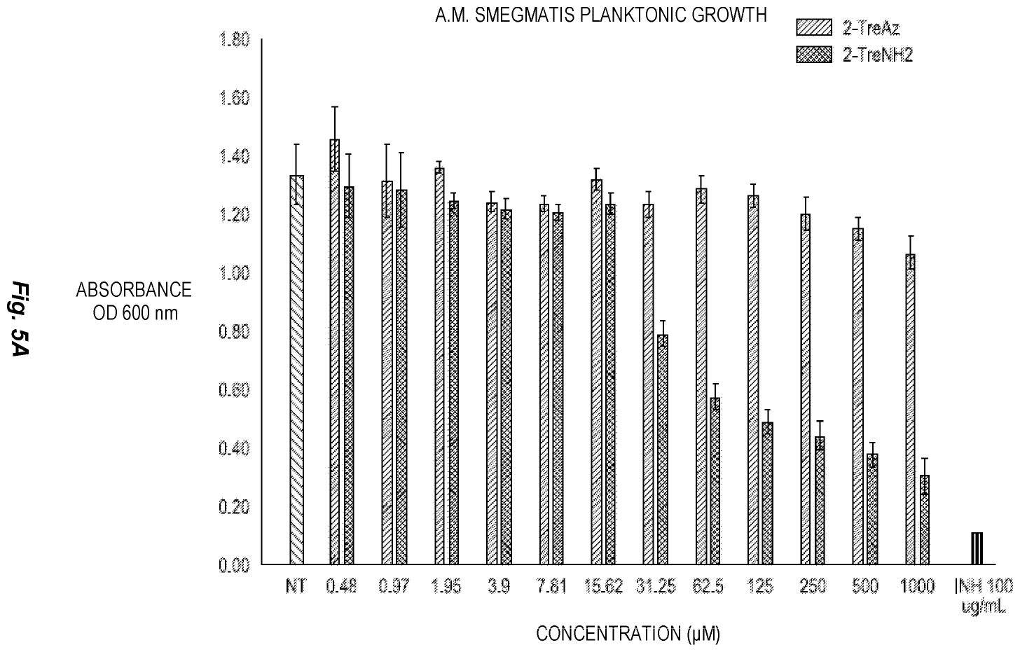

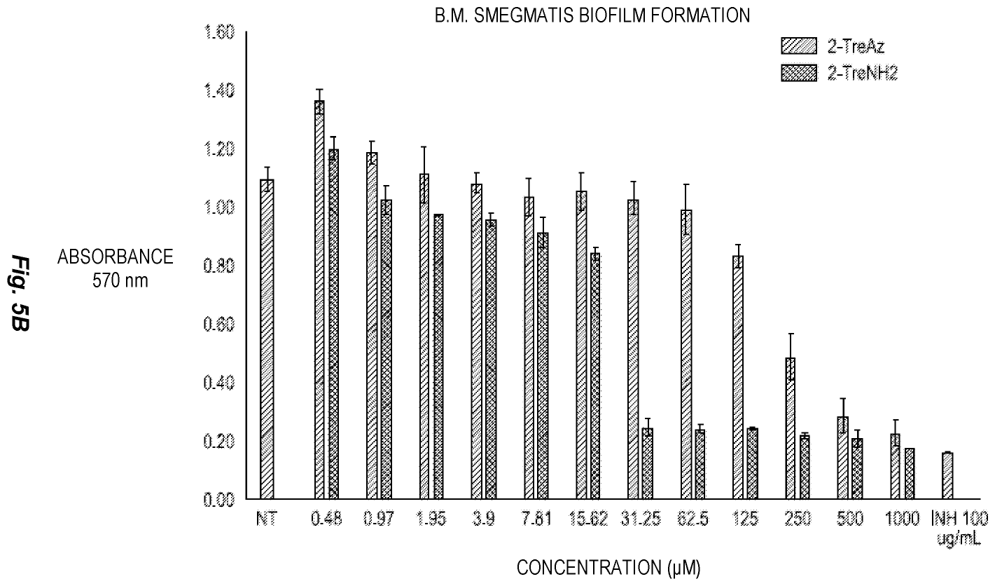

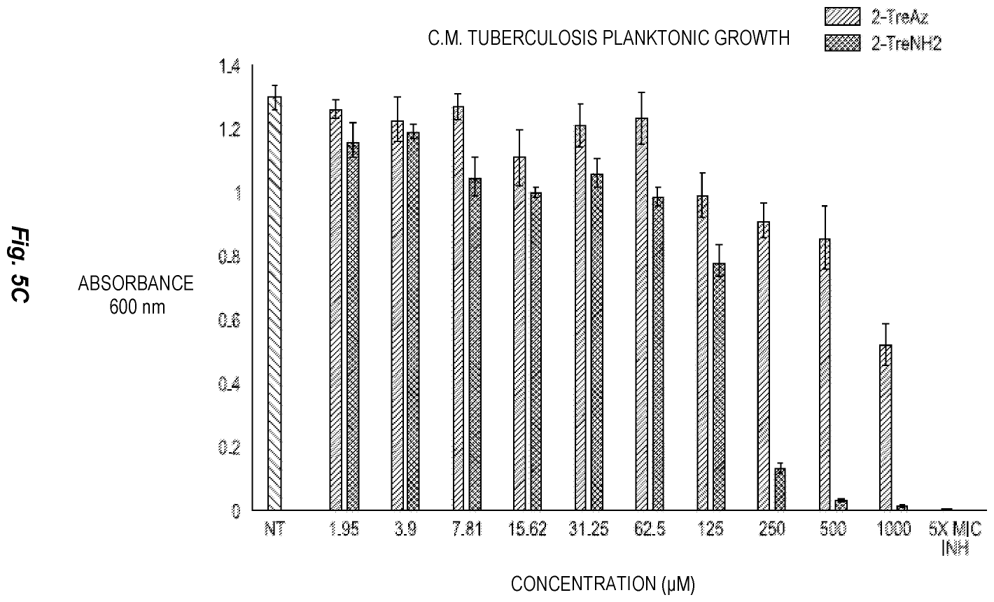

The study also showed that treatment with 6-treAz sensitized M. tuberculosis to an existing clinical anti-tubercular compound, bedaquiline. To determine whether 6-treAz could be a potential candidate for adjunctive therapeutic intervention, we treated WT and ΔtreS with 100 μM 6-treAz and 10×MIC (minimal inhibitory concentration) equivalent bedaquiline for 9 days and observed that WT M. tuberculosis displayed rapid and heightened sensitivity to bedaquiline by 100 CFU, a level similar to that of ΔtreS (FIG. 3). Intriguingly, the metabolome profile of WT M. tuberculosis after treatment with 10×MIC BDQ and/or INH was similar to that of Δtres (FIG. 4), suggesting that enhanced antibiotic effect of BDQ after treatment with 6-treAz was attributed to lack of TreS activity and accompanied deficiency in the trehalose catalytic shift.

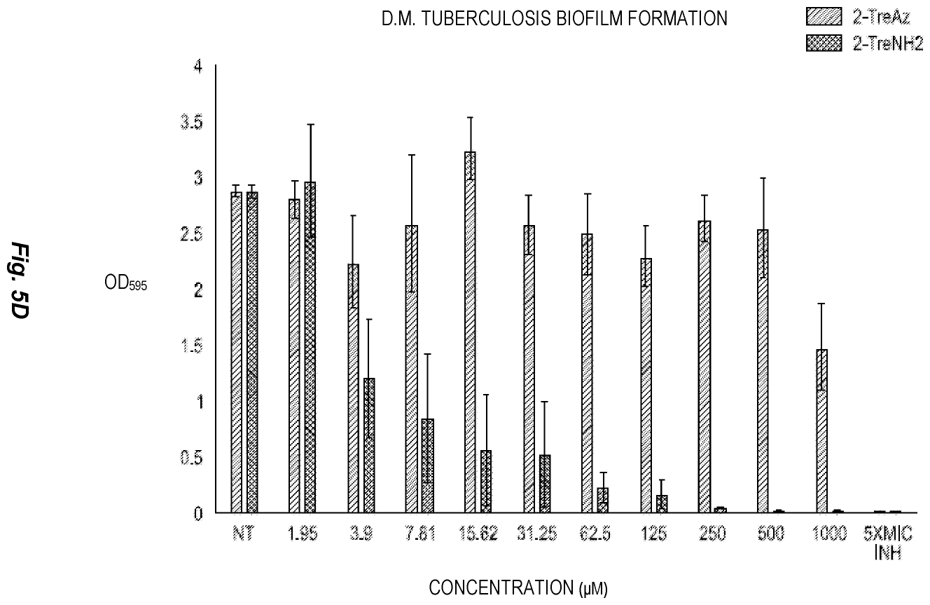

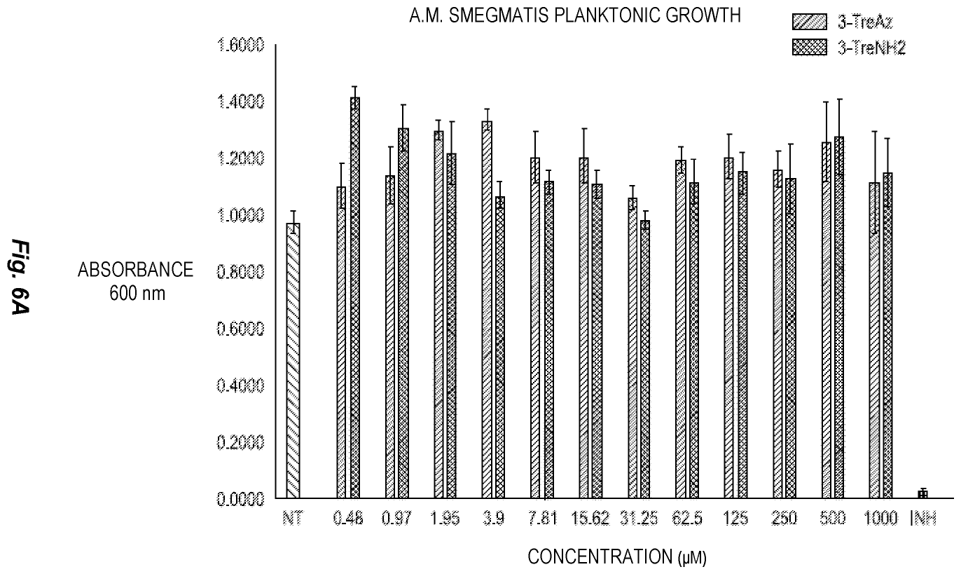

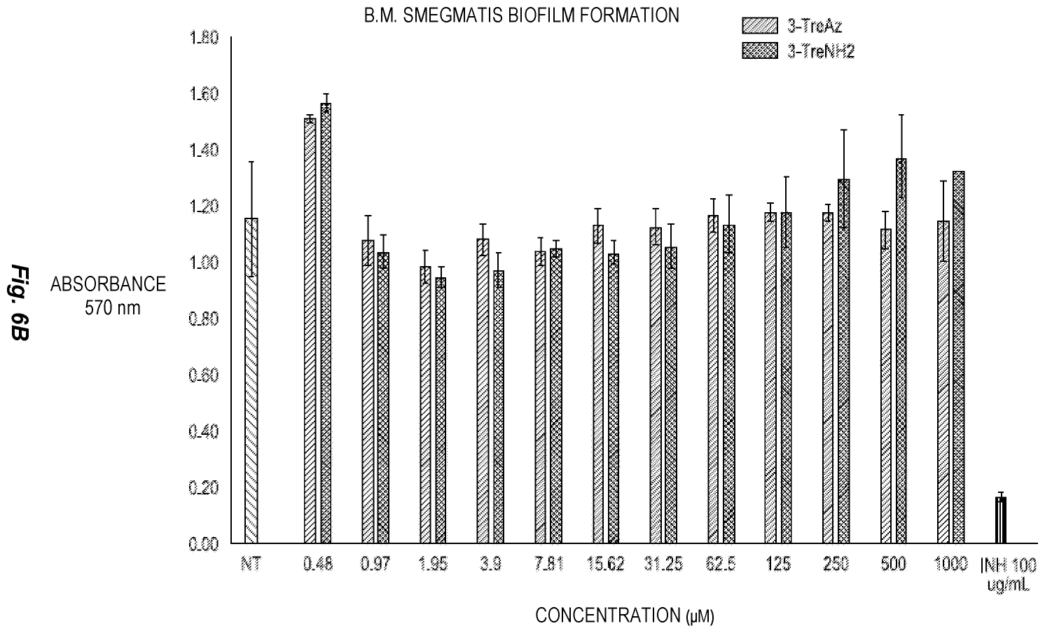

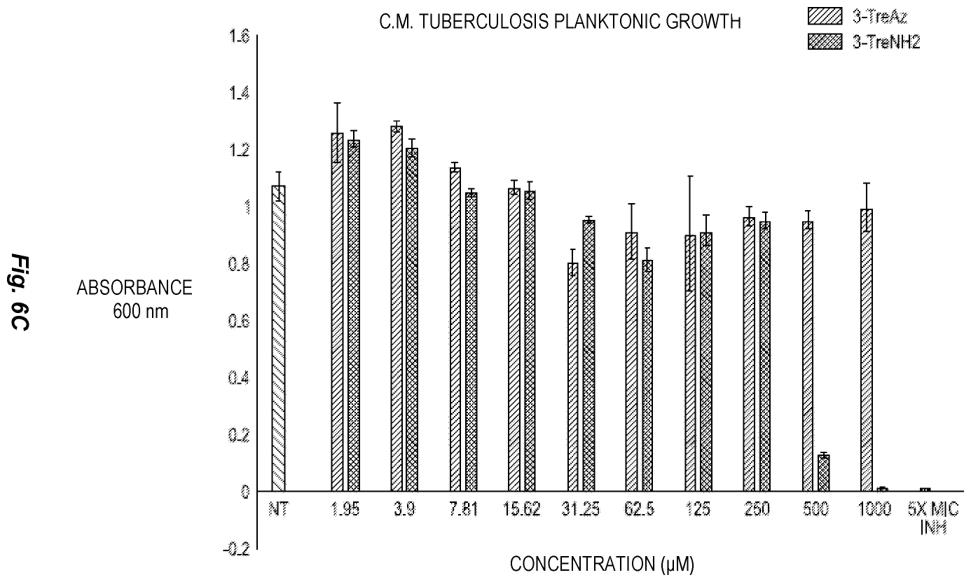

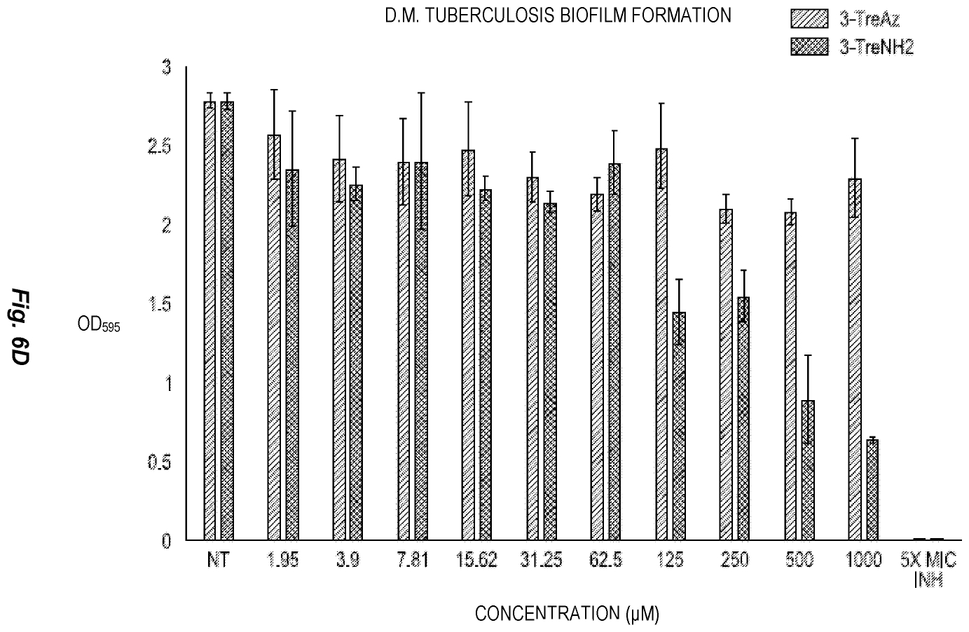

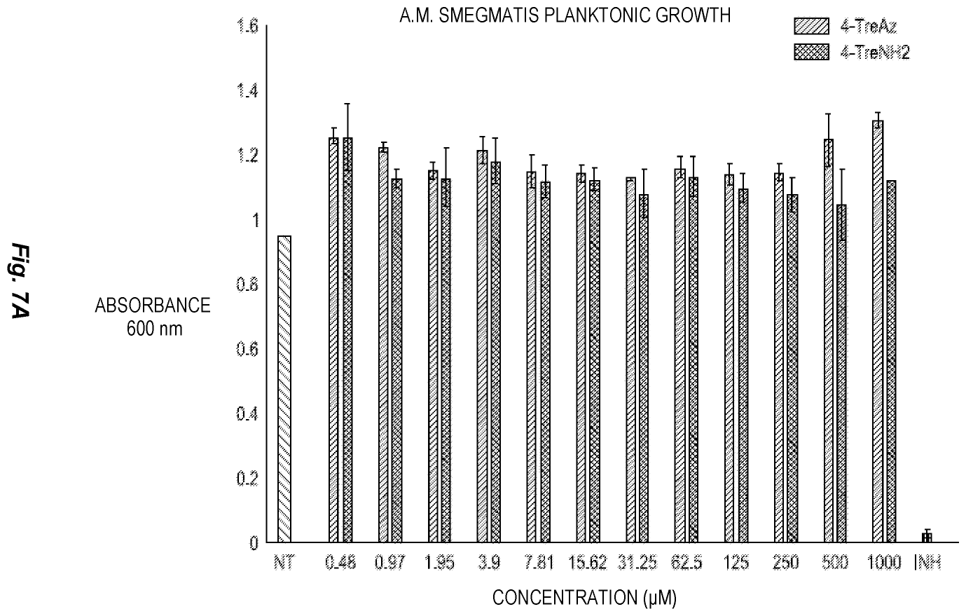

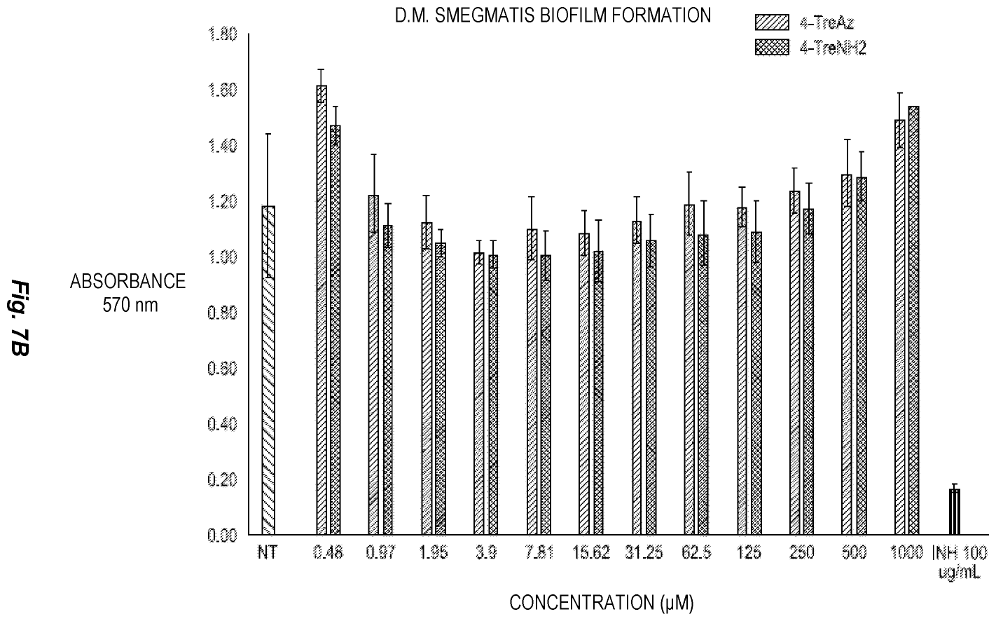

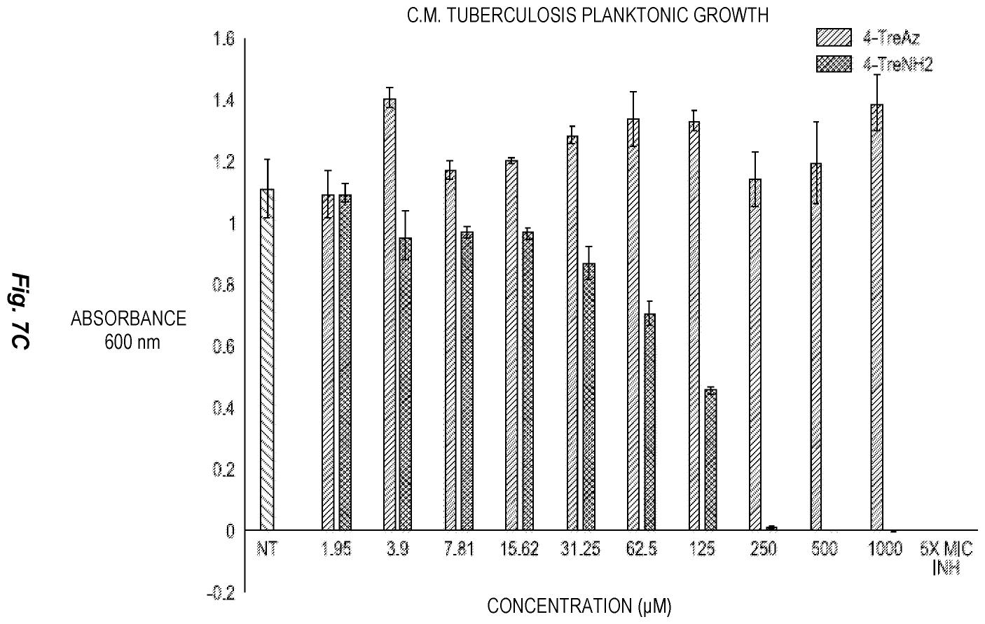

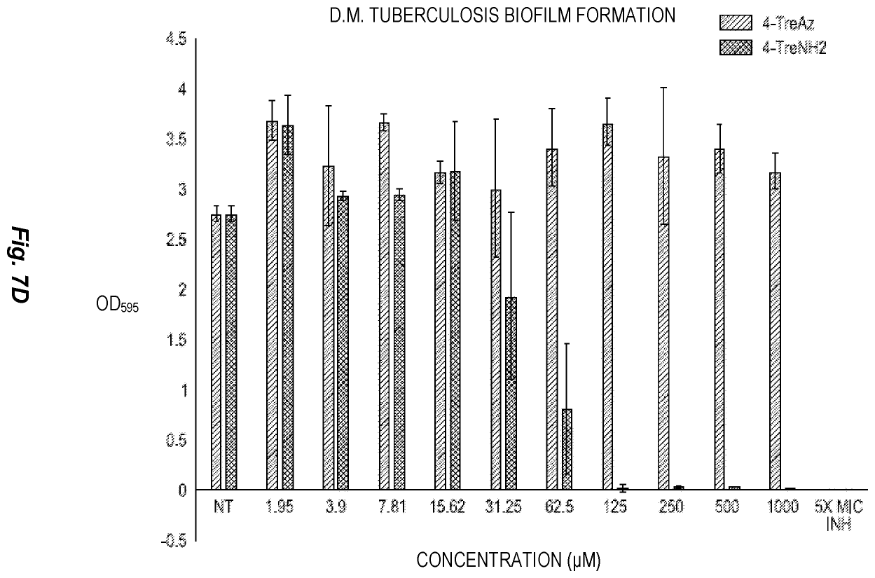

To identify inhibitors with improved properties, our team systematically investigated other azidodeoxy and aminodeoxy trehalose analogues for activity against M. tuberculosis planktonic growth, biofilm formation, and the trehalose catalytic shift. Using synthetic methods previously developed, we synthesized the panel of 8 azidodeoxy and aminodeoxy compounds shown in Chart 1 and two epimers (Scheme 2) to enable a systematic evaluation. We used our reported methods to evaluate planktonic growth and biofilm inhibition of a non-pathogenic model organism, M. smegmatis, and global pathogen, M. tuberculosis. Data for all compounds are shown in FIGS. 5-8 in order of the position of modification on trehalose. The novel findings from these experiments are:
2-TreNH₂ was shown for first time to inhibit M. smegmatis and M. tuberculosis biofilm formation.
3-TreNH₂ was shown for first time to be a moderate inhibitor of M. tuberculosis biofilm formation but not M. smegmatis biofilm formation.
4-TreNH₂ shown for first time to inhibit M. tuberculosis biofilm formation; similar potency to 2-TreNH₂; and did not exhibit activity against M. smegmatis (4-TreNH₂ has never been previously tested against M. tuberculosis because it did not have activity against M. smegmatis).

It is noted that 2-, 3- and 4-TreNH₂ are natural products and can in principle be produced on industrial scale by biocatalysis, increasing the relevance of preceding points for drug development.

Chart 1. Structures of (A) Azidodeoxy and (B) Aminodeoxy Trehalose Compounds*. *2-epi-TreAz, 2-TreAz, and 4-TreAz are advanced drug candidates that can kill mycobacterial persisters and boost efficacy of known TB antibiotics. 2-epi-TreAz appears based upon preliminary results to be the most potent. The biofilm inhibition assay, normal growth inhibition assay, and antibiotic synergism assay indicated that, overall, 2-TreAz is a top candidate, whereas 4-TreAz did not show activity against M. smegmatis or M. tuberculosis.

### A. Azidodeoxy Trehalose Compounds

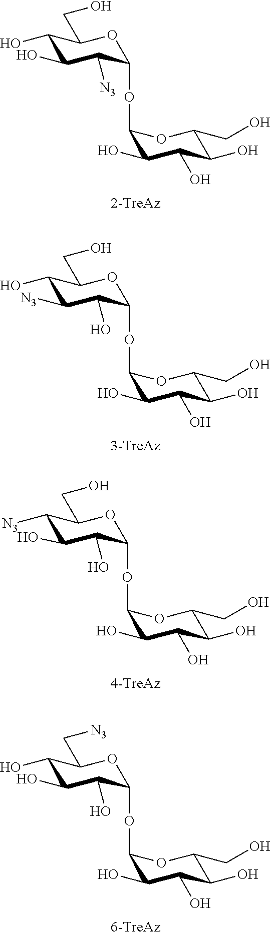

### B. Aminodeoxy Trehalose Compounds

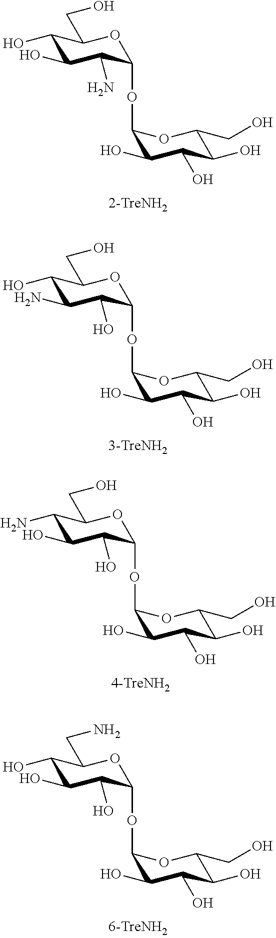

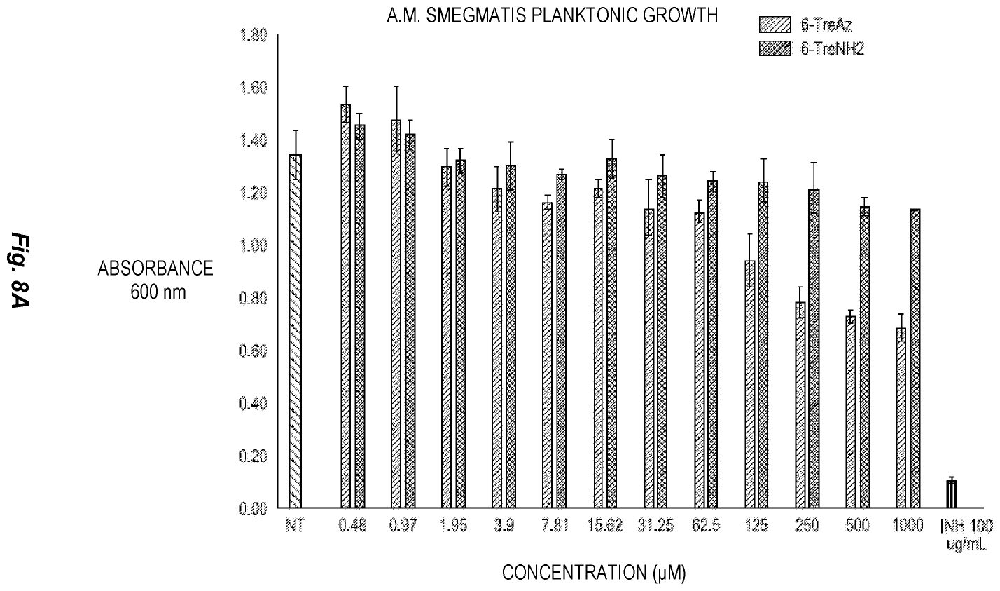

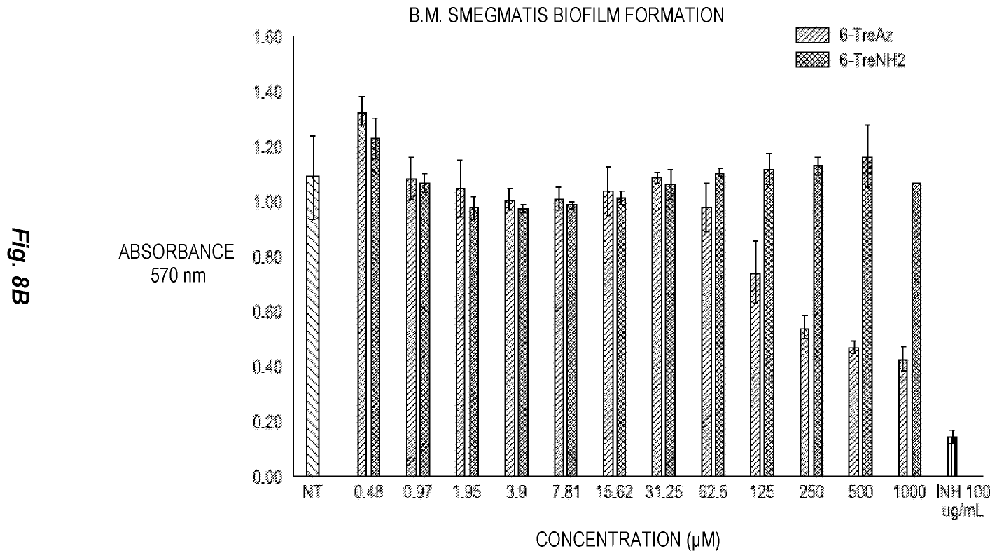

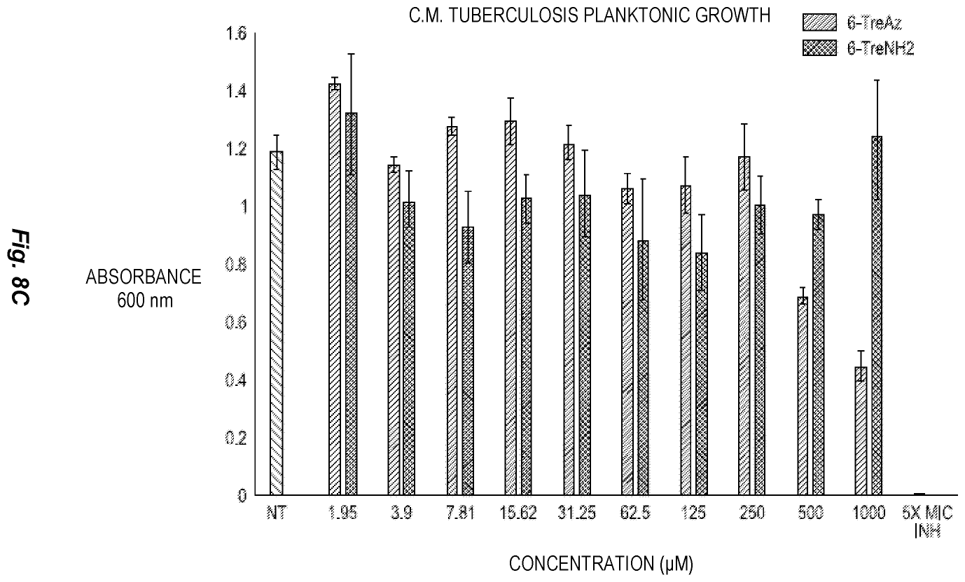

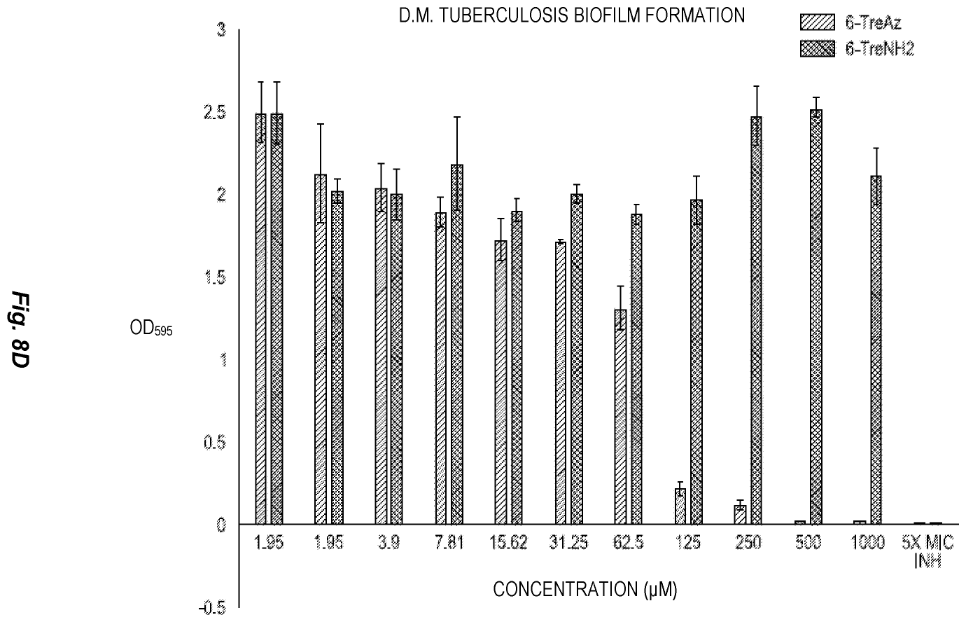

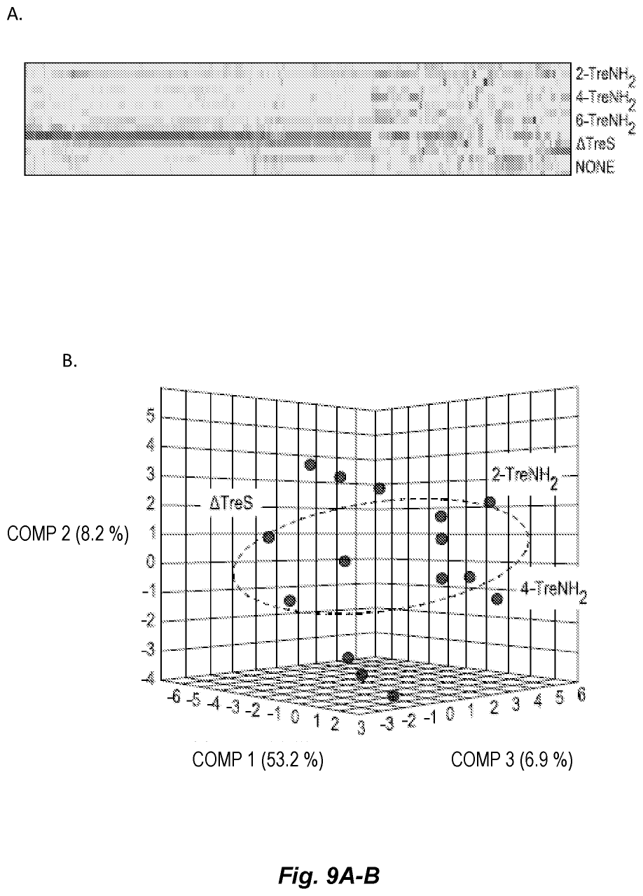

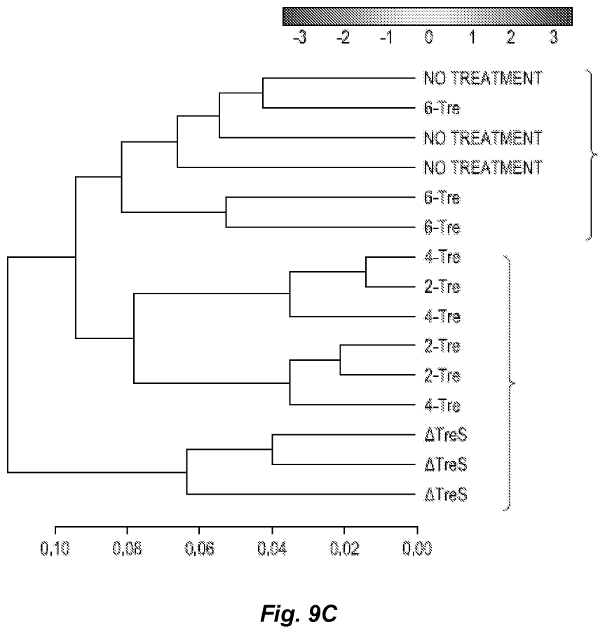

To identify that newly synthesized aminodeoxy trehalose analogues as TreS-specific inhibitors, we characterized M. tuberculosis metabolomics by profiling and comparing them with that of ΔtreS, a M. tuberculosis mutant lacking the trehalose catalytic shift. Metabolomics remodeling caused by treatment with 2-, 4-, and 6-TreNH₂ was determined by comparing the relative abundance of ˜410 known metabolites. Using Metaboanalyst (ver. 5.0) bioinformatic tools for multivariate hierarchical clustering analysis, principal component analysis, and molecular phylogeny, the analyses indicated that the metabolic alterations occurring in M. tuberculosis after treatment with 2- and 4-TreNH₂ were relatively similar to ΔtreS (FIG. 9). However, 6-TreNH₂ treated M. tuberculosis is metabolically different from ΔtreS (FIG. 9). The phenotypic and metabolic outcomes enable us to conclude that 2- and 4-TreNH₂ are potential candidates of M. tuberculosis trehalose catalytic shift inhibitors, a source of adjunctive TB therapeutic interventions. In addition, 6-treNH₂ serves as a negative control for further structural analysis.

From experimental observations, we discovered, 1) 6-TreNH₂ has no inhibitory effect on M. tuberculosis planktonic growth as well as on biofilm formation; and 2) 2- and 4-TreNH₂ have an impact on the M. tuberculosis metabolome, which was similar to that of ΔtreS, a TreS-deficient M. tuberculosis. Intriguingly, 6-TreNH₂-mediated M. tuberculosis metabolome was metabolically unrelated to that of ΔtreS, and rather more similar to that of wild type M. tuberculosis.

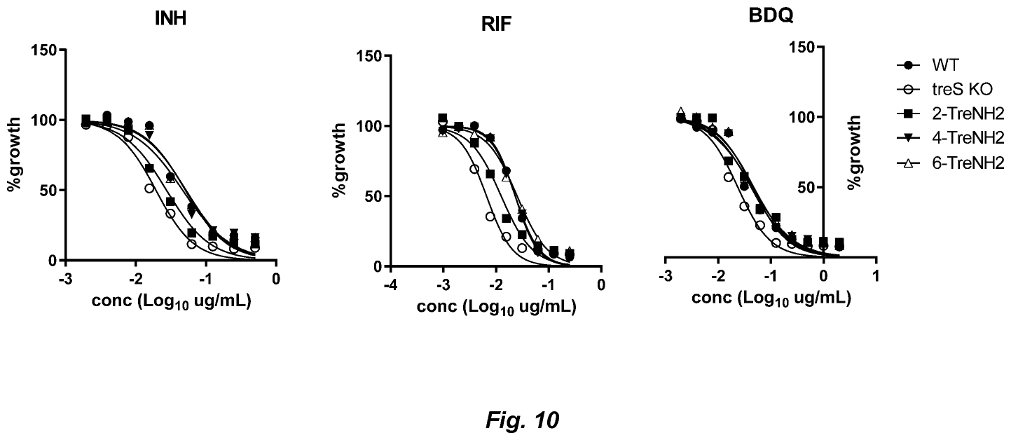

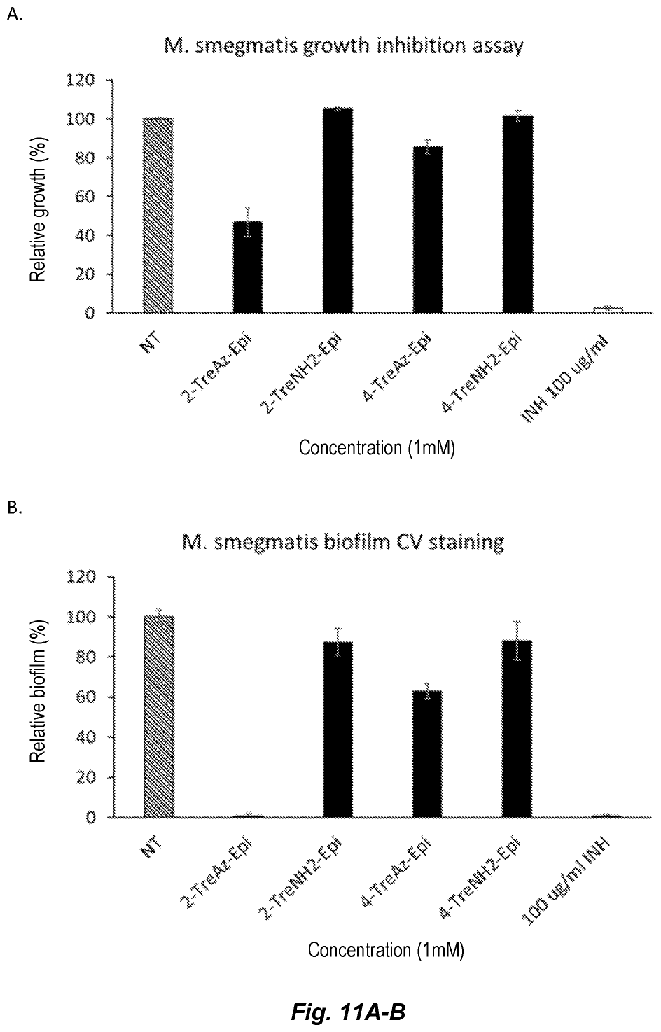

To identify the antimycobacterial synergy of aminodeoxy trehalose analogues with known TB antibiotics, we conducted an IC₉₅ shift assay using M. tuberculosis. ΔtreS was included as a positive control (FIG. 10). Intriguingly, co-treatment with 2-TreNH₂ and isoniazid (INH) or rifampicin (RIF), not bedaquiline (BDQ), significantly reduced the IC₉₅ values against M. tuberculosis. Under all conditions, ΔtreS showed the biggest range of IC₉₅ value shift (Table 1). Consistent with our phenotypic analysis and metabolomics analysis, 6-TreNH₂ showed no effect on antimycobacterial activity of TB antibiotics (FIG. 10). The most potent compound, 6-TreNH₂, showed a weaker antimycobacterial synergy activity compared to that of ΔtreS, suggesting that there is room to improve the affinity between 6-TreNH₂ and a trehalose binding site of the TreS enzyme. Key points are summarized below:
2-TreNH₂ is the compound that improved the antimycobacterial effects of known first-line TB antibiotics.
6-TreNH₂ has no impact on antibiotic effects of INH or RIF against replicating M. tuberculosis.
2-TreNH₂ is a potential compound that can be used as an adjunctive therapy with known TB antibiotics.

TABLE 1
Drug resistance phenotypes (MIC shift assay) for
H37Rv lab strain in the presence of varying doses of anti-TB
antibiotics with and without trehalosamine compounds.

| IC₉₅ (ug/mL) | WT | ΔtreS | 2-treNH₂ | 4-treNH₂ | 6-treNH₂ |
|---|---|---|---|---|---|
| INH | 0.28 | 0.12 | 0.21 | 0.44 | 0.33 |
| RIF | 0.18 | 0.025 | 0.09 | 0.14 | 0.24 |
| BDQ | 0.59 | 0.21 | 0.78 | 0.39 | 0.57 |

The synthesis of the new trehalose structural analogues, 2-epi-TreAz and 2-epi-TreNH₂ is shown in Scheme 2. It shows a simple one step synthesis strategy of 2-epi-TreAz using a chemoenzymatic method. 2-Epi-TreAz can inhibit planktonic growth and biofilm formation of M. tuberculosis.

Additionally, the synthesis of compounds 2-TreAz and 2-TreNH₂ shown in Scheme 3 were prepared from the common intermediate, compound 6.

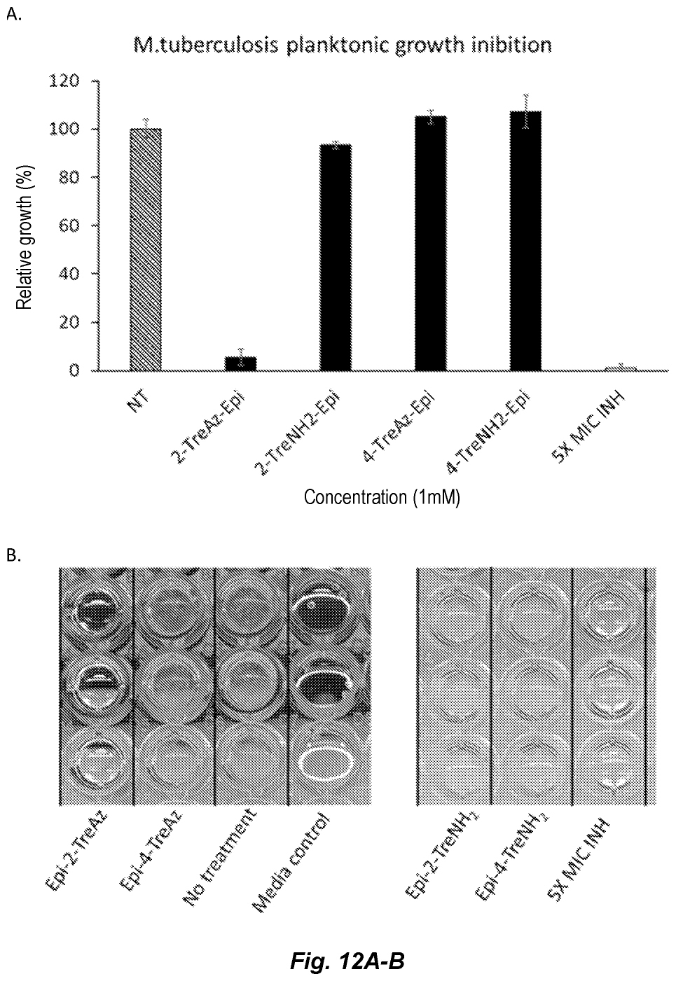

FIG. 12 shows that 2-epi-TreAz selectively blocks biofilm formation in M. smegmatis at 1 mM. FIG. 13 shows that 2-epi-TreAz also completely blocks planktonic growth of M. tuberculosis at 1 mM and that 2-epi-TreAz completely blocks biofilm formation of M. tuberculosis at 1 mM.

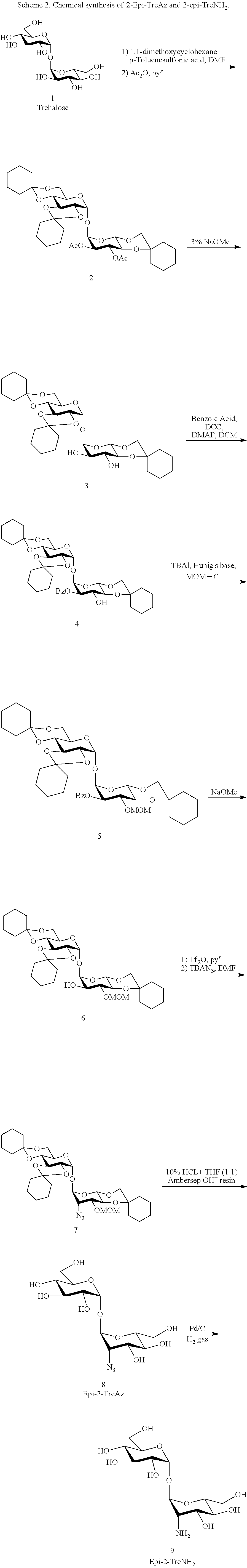

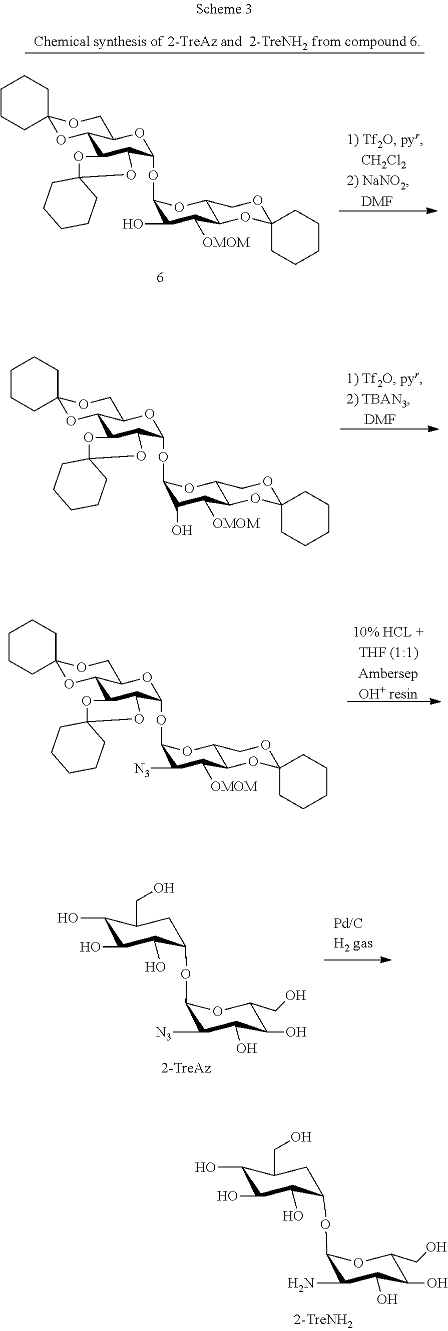

### Pharmaceutical Formulations.

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The compounds described herein can be effective anti-bacterial agents and have higher potency in combination with other anti-bacterial agents.

The invention provides therapeutic methods of treating tuberculosis in a mammal, which involve administering to a mammal having tuberculosis an effective amount of a compound or composition described herein. A mammal includes a primate, human, rodent, canine, feline, bovine, ovine, equine, swine, caprine, bovine and the like.

The ability of a compound of the invention to treat tuberculosis may be determined by using assays well known to the art. For example, known treatment protocols, toxicity evaluations, data analysis methods, and quantification of bacterial cell kill.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Synthesis of Compounds

2-TreAz and 2-TreNH₂ were prepared by chemoenzymatic synthesis using a reported method (J. Org. Chem. 2018, 83, 8662-8667). 3- and 6-TreAz were prepared by chemoenzymatic synthesis using a reported method (ChemBioChem 2014 15, 2066-2070). To access 3- and 6-TreNH₂, 3- and 6-TreAz respectively were subjected to Pd-catalyzed hydrogenation as previously described (J. Am. Chem. Soc. 2017, 139, 3488-3495). 4-TreAz and 4-TreNH₂ were prepared by chemical synthesis using a reported method (Carbohyd. Res. 1993, 239, 197-207). NMR spectra for all compounds matched those previously reported.

The compound 4-TreNH₂ can be produced on industrial scale. Additional information and data supporting the synthesis is described by Wada et al., Adv. Biology 2022, 2101309 (DOI: 10.1002/adbi.202101309) and its Supporting Information, which is incorporated herein by reference in its entirety.

### Example 2. Biological Assays

Planktonic growth and biofilm assays. Evaluation of planktonic growth and biofilm formation inhibition for M. smegmatis was carried out as previously reported for other trehalose analogues (Carbohydr. Res. 2017, 450, 60-66). Evaluation of planktonic growth and biofilm formation inhibition for M. tuberculosis was carried out as previously reported for 6-TreAz (Nat. Commun., 2019, 2928-2928).

Drug sensitivity test. CFU-based cell enumeration assays were conducted in 96 well plates. Mid-log phase M. tuberculosis H37Rv in the absence or presence of various amount of trehalose analogues was diluted. Known TB antibiotics (INH or BDQ) were added at the indicated concentrations and incubated. The cultures were serially diluted and plated on Middlebrook 7H10 agar, incubated for 3 additional weeks at 37° C., and colonies were counted. In all inhibition experiments, the negative control was untreated bacteria and the positive control was treatment with known antibiotics at concentrations greater than the minimum inhibitory concentration (MIC). Data are the average of three replicate experiments and error bars represent standard deviation. Data are representative of at least two independent experiments.

In vitro TreS assay. TreS activity was measured using a 100 μL in vitro enzyme reaction containing 40 mM MOPS (pH 7.0), 10 mM trehalose, and 20 ng of purified TreS enzyme in the presence or absence of 6-treAz or validamycin A (a known TreS inhibitor). The reaction was initiated by adding TreS enzyme, incubated at 37° C., and then quenched by heating and adding acetonitrile containing 0.2% formic acid. After centrifugation, supernatants were used for LC-MS to quantify maltose product.

Metabolomics profiling. Metabolome extraction, detection, quantification, and analysis were carried out as previously reported (Proc Natl Acad Sci USA 2013, 110, 6554-6559). Extracted metabolites were separated on Cogent Diamond Hydride Type C column (gradient 3) and the mobile phase consisted of sol A (ddH₂O with 0.2% formic acid and sol B (acetonitrile with 0.2% formic acid). The mass spectrometer used was an Agilent Accurate Mass 6230 time of flight (TOF) coupled with an Agilent 1290 liquid chromatography (LC) system. Detected ions were deemed metabolites on the basis of unique accurate mass retention time identifiers for masses exhibiting the expected distribution of accompanying isotopologues. The abundance of metabolites was extracted using Agilent Qualitative Analysis B.07.00 and Profinder B.08.00 software (Agilent Technologies) with a mass tolerance of <0.005 Da.

Antibacterial activity of the compounds. All compounds were dissolved in DMSO and dispensed using a HP D300e Digital Dispensor in a 96 well plate format. DMSO didn't exceed 1% of the final culture volume and was maintained at the same concentration across all samples. H37Rv Mtb strain were growth-synchronized for the MIC shift assay. Cultures were then back diluted to a starting OD₅₉₅ of 0.05 and 50 μL of cell suspension was plated in technical triplicate in wells containing the test compounds and anti-TB antibiotics such as bedaquiline (BDQ), rifampicin (RIF), and isoniazid (INH). For checkerboard assay and MIC shift assay were growth-synchronized to late log-phase and back-diluted to an OD₅₉₅ of 0.025 prior to plating. Plates were incubated at 37° C. with 5% CO₂. OD₅₉₅ was evaluated using a plate reader at 10-14 days post-plating and percent growth was calculated relative to the vehicle control for each condition. IC₅₀ measurements were calculated using a non-linear fit in GraphPad Prism.

To quantify growth phenotypes on Middlebrook 7H10 (m⁷H10) agar plate, 10-fold serial dilutions of the Mtb cultures were spotted on m⁷H10 agar plate containing antibiotics at the indicated concentrations and/or trehalose analogue compounds. Plates were incubated at 37° C. for 20 days.

### Example 3. Pharmaceutical Dosage Forms

The following formulations illustrate representative pharmaceutical dosage forms that may be used for the therapeutic or prophylactic administration of a compound or composition of a formula described herein, a composition specifically disclosed herein, or a pharmaceutically acceptable salt or solvate thereof (hereinafter referred to as ‘Composition X’):

| (i) Tablet 1 | mg/tablet |
|---|---|
| ‘Composition X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Composition X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Composition X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Composition X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Composition X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 Ml |

| (vi) Aerosol | mg/can |
|---|---|
| ‘Composition X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Composition X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Composition X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Composition X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Composition X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Composition X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Composition X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method for treating a mycobacterial disease in a subject in need thereof comprising administering to the subject a therapeutically effective amount of a mycobacterial antibiotic and a therapeutically effective amount of one or more deoxy trehaloses that are capable of sensitizing mycobacteria to the mycobacterial antibiotic, wherein the mycobacterial disease in the subject is thereby treated.

2. The method of claim 1 wherein the one or more deoxy trehaloses comprise one or more aminodeoxy trehaloses, one or more azidodeoxy trehaloses, or a combination thereof.

3. The method of claim 2 wherein the one or more deoxy trehaloses is one or more aminodeoxy trehaloses represented by: 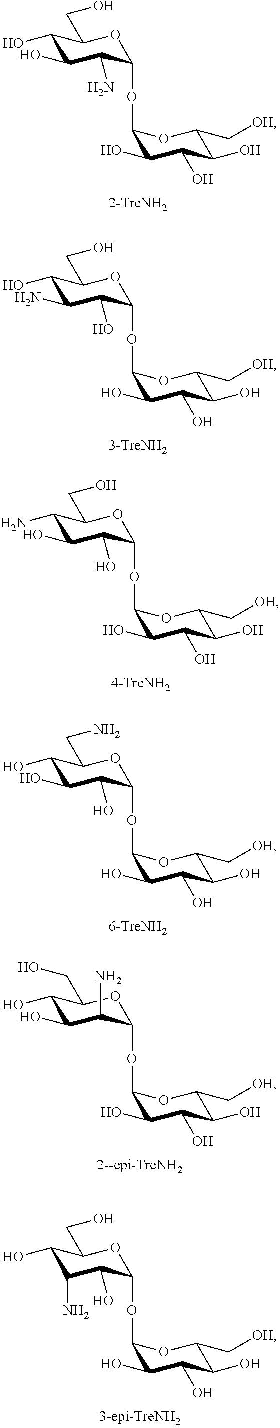 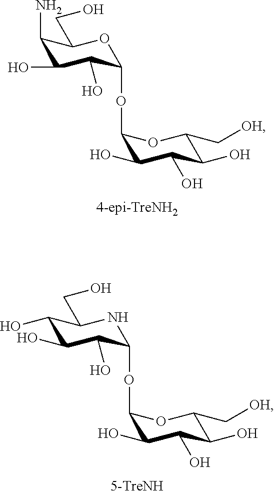
or a combination thereof.

4. The method of claim 2 wherein the one or more deoxy trehaloses is one or more azidodeoxy trehaloses represented by: 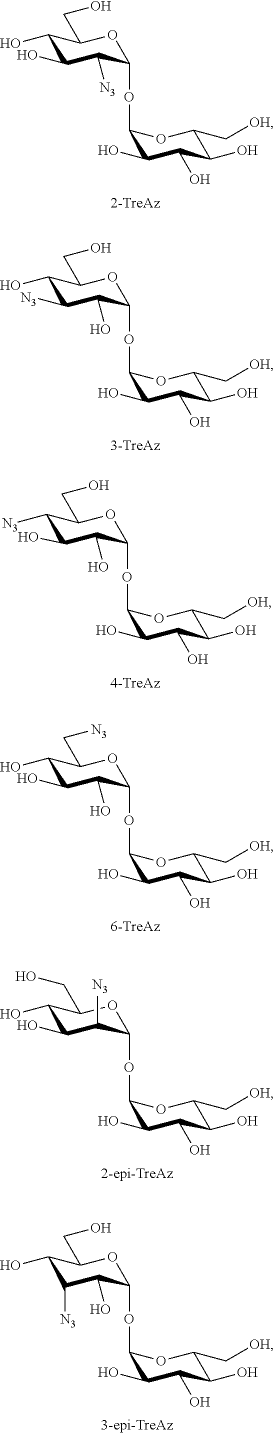 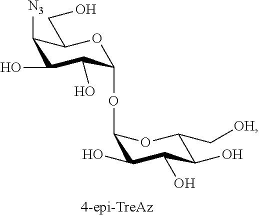
or a combination thereof.

5. The method of claim 1 wherein the mycobacterial disease is tuberculosis or leprosy.

6. The method of claim 1 wherein the mycobacterial antibiotic is bedaquiline, ethambutol, isoniazid, pyrazinamide, rifampicin, streptomycin, ciprofloxacin, moxifloxacin, p-aminosalicylic acid, or a combination thereof; or
wherein the mycobacteria is Mycobacterium tuberculosis, Mycobacterium leprae, Mycobacterium smegmatis, Mycobacterium aurum, Mycobacterium avium, Mycobacterium abscessus, or Mycobacterium fortuitum.

7. The method of claim 1 wherein the therapeutically effective amount of the one or more deoxy trehaloses is a systemic concentration of about 1 micromolar to about 1000 micromolar of the one or more deoxy trehaloses at steady state in the subject; or
wherein the therapeutically effective amount of the mycobacterial antibiotic is a systemic concentration of about 1 nanomolar to about 1000 micromolar of the mycobacterial antibiotic at steady state in the subject.

8. A method for inhibiting mycobacterial growth, mycobacterium planktonic growth, or mycobacterial biofilm formation comprising contacting mycobacteria, an effective amount of a mycobacterial antibiotic, and an effective amount of one or more deoxy trehaloses, wherein inhibition of mycobacterial growth, mycobacterium planktonic growth, or mycobacterial biofilm formation occurs by inhibition of a trehalose-catalytic shift in a biosynthetic pathway of the mycobacteria.

9. The method of claim 8 wherein the one or more deoxy trehaloses comprise one or more aminodeoxy trehaloses, one or more azidodeoxy trehaloses, or a combination thereof.

10. The method of claim 9 wherein the one or more deoxy trehaloses is:
(2R,3R,4S,5S,6R)-2-(((2R,3R,4R,5S,6R)-3-azido-4,5-dihydroxy-6-(hydroxymethyl)tetrahydro-2H-pyran-2-yl)oxy)-6-(hydroxymethyl)tetrahydro-2H-pyran-3,4,5-triol (2-TreAz),
(2R,3R,4S,5S,6R)-2-(((2R,3R,4S,5S,6R)-4-azido-3,5-dihydroxy-6-(hydroxymethyl)tetrahydro-2H-pyran-2-yl)oxy)-6-(hydroxymethyl)tetrahydro-2H-pyran-3,4,5-triol (3-TreAz),
(2R,3R,4S,5S,6R)-2-(((2R,3R,4S,5S,6S)-5-azido-3,4-dihydroxy-6-(hydroxymethyl)tetrahydro-2H-pyran-2-yl)oxy)-6-(hydroxymethyl)tetrahydro-2H-pyran-3,4,5-triol (4-TreAz),
(2R,3S,4S,5R,6R)-2-(azidomethyl)-6-(((2R,3R,4S,5S,6R)-3,4,5-trihydroxy-6-(hydroxymethyl)tetrahydro-2H-pyran-2-yl)oxy)tetrahydro-2H-pyran-3,4,5-triol (6-TreAz),
(2R,3R,4S,5S,6R)-2-(((2R,3S,4R,5S,6R)-3-azido-4,5-dihydroxy-6-(hydroxymethyl)tetrahydro-2H-pyran-2-yl)oxy)-6-(hydroxymethyl)tetrahydro-2H-pyran-3,4,5-triol (2-epi-TreAz),
(2R,3R,4S,5S,6R)-2-(((2R,3R,4R,5S,6R)-4-azido-3,5-dihydroxy-6-(hydroxymethyl)tetrahydro-2H-pyran-2-yl)oxy)-6-(hydroxymethyl)tetrahydro-2H-pyran-3,4,5-triol (3-epi-TreAz),
(2R,3R,4S,5S,6R)-2-(((2R,3R,4S,5R,6S)-5-azido-3,4-dihydroxy-6-(hydroxymethyl)tetrahydro-2H-pyran-2-yl)oxy)-6-(hydroxymethyl)tetrahydro-2H-pyran-3,4,5-triol (4-epi-TreAz),
(2R,3R,4S,5S,6R)-2-(((2R,3R,4R,5S,6R)-3-amino-4,5-dihydroxy-6-(hydroxymethyl)tetrahydro-2H-pyran-2-yl)oxy)-6-(hydroxymethyl)tetrahydro-2H-pyran-3,4,5-triol (2-TreNH₂),
(2R,3R,4S,5S,6R)-2-(((2R,3R,4S,5S,6R)-4-amino-3,5-dihydroxy-6-(hydroxymethyl)tetrahydro-2H-pyran-2-yl)oxy)-6-(hydroxymethyl)tetrahydro-2H-pyran-3,4,5-triol (3-TreNH₂),
(2R,3R,4S,5S,6R)-2-(((2R,3R,4S,5S,6S)-5-amino-3,4-dihydroxy-6-(hydroxymethyl)tetrahydro-2H-pyran-2-yl)oxy)-6-(hydroxymethyl)tetrahydro-2H-pyran-3,4,5-triol (4-TreNH₂),
(2R,3S,4S,5R,6R)-2-(aminomethyl)-6-(((2R,3R,4S,5S,6R)-3,4,5-trihydroxy-6-(hydroxymethyl)tetrahydro-2H-pyran-2-yl)oxy)tetrahydro-2H-pyran-3,4,5-triol (6-TreNH₂),
(2R,3R,4S,5S,6R)-2-(((2R,3S,4R,5S,6R)-3-amino-4,5-dihydroxy-6-(hydroxymethyl)tetrahydro-2H-pyran-2-yl)oxy)-6-(hydroxymethyl)tetrahydro-2H-pyran-3,4,5-triol (2-epi-TreNH₂),
(2R,3R,4S,5S,6R)-2-(((2R,3R,4R,5S,6R)-4-amino-3,5-dihydroxy-6-(hydroxymethyl)tetrahydro-2H-pyran-2-yl)oxy)-6-(hydroxymethyl)tetrahydro-2H-pyran-3,4,5-triol (3-epi-TreNH₂),
(2R,3R,4S,5S,6R)-2-(((2R,3R,4S,5R,6S)-5-amino-3,4-dihydroxy-6-(hydroxymethyl)tetrahydro-2H-pyran-2-yl)oxy)-6-(hydroxymethyl)tetrahydro-2H-pyran-3,4,5-triol (4-epi-TreNH₂),
(2R,3R,4S,5R,6R)-2-(hydroxymethyl)-6-(((2R,3R,4S,5S,6R)-3,4,5-trihydroxy-6-(hydroxymethyl)tetrahydro-2H-pyran-2-yl)oxy) piperidine-3,4,5-triol (5-TreNH), or
a combination thereof.

11. The method of claim 8 wherein the mycobacterial antibiotic is bedaquiline, ethambutol, isoniazid, pyrazinamide, rifampicin, streptomycin, ciprofloxacin, moxifloxacin, p-aminosalicylic acid, or a combination thereof, or
wherein the mycobacteria is Mycobacterium tuberculosis, Mycobacterium leprae, Mycobacterium smegmatis, Mycobacterium aurum, Mycobacterium avium, Mycobacterium abscessus, or Mycobacterium fortuitum.

12. The method of claim 8 wherein the effective amount of the one or more deoxy trehaloses and/or the effective amount of the mycobacterial antibiotic is at a concentration of about 1 nanomolar to about 1000 micromolar of the one or more deoxy trehaloses and/or the mycobacterial antibiotic.

13. A composition comprising a mycobacterial antibiotic and one or more deoxy trehaloses of Formula I: 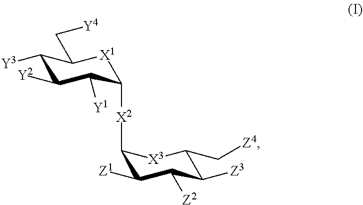
or an epimer thereof;
wherein
X¹, X², and X³ are each independently O or NR¹;
Y¹, Y², Y³ and Y⁴ are each independently OR², N₃, or N(R¹)₂;
Z¹, Z², Z³ and Z⁴ are each independently OR², N₃, or N(R¹)₂; and
R¹ and R² are each independently H or (C₁-C₆)alkyl;
wherein each of the one or more deoxy trehaloses of Formula I comprises at least one N₃, or N(R¹)₂ moiety.

14. The composition of claim 13 wherein the one or more deoxy trehaloses is:
(2R,3R,4S,5S,6R)-2-(((2R,3R,4R,5S,6R)-3-azido-4,5-dihydroxy-6-(hydroxymethyl)tetrahydro-2H-pyran-2-yl)oxy)-6-(hydroxymethyl)tetrahydro-2H-pyran-3,4,5-triol (2-TreAz),
(2R,3R,4S,5S,6R)-2-(((2R,3R,4S,5S,6R)-4-azido-3,5-dihydroxy-6-(hydroxymethyl)tetrahydro-2H-pyran-2-yl)oxy)-6-(hydroxymethyl)tetrahydro-2H-pyran-3,4,5-triol (3-TreAz),
(2R,3R,4S,5S,6R)-2-(((2R,3R,4S,5S,6S)-5-azido-3,4-dihydroxy-6-(hydroxymethyl)tetrahydro-2H-pyran-2-yl)oxy)-6-(hydroxymethyl)tetrahydro-2H-pyran-3,4,5-triol (4-TreAz),
(2R,3S,4S,5R,6R)-2-(azidomethyl)-6-(((2R,3R,4S,5S,6R)-3,4,5-trihydroxy-6-(hydroxymethyl)tetrahydro-2H-pyran-2-yl)oxy)tetrahydro-2H-pyran-3,4,5-triol (6-TreAz),
(2R,3R,4S,5S,6R)-2-(((2R,3S,4R,5S,6R)-3-azido-4,5-dihydroxy-6-(hydroxymethyl)tetrahydro-2H-pyran-2-yl)oxy)-6-(hydroxymethyl)tetrahydro-2H-pyran-3,4,5-triol (2-epi-TreAz),
(2R,3R,4S,5S,6R)-2-(((2R,3R,4R,5S,6R)-4-azido-3,5-dihydroxy-6-(hydroxymethyl)tetrahydro-2H-pyran-2-yl)oxy)-6-(hydroxymethyl)tetrahydro-2H-pyran-3,4,5-triol (3-epi-TreAz),
(2R,3R,4S,5S,6R)-2-(((2R,3R,4S,5R,6S)-5-azido-3,4-dihydroxy-6-(hydroxymethyl)tetrahydro-2H-pyran-2-yl)oxy)-6-(hydroxymethyl)tetrahydro-2H-pyran-3,4,5-triol (4-epi-TreAz),
(2R,3R,4S,5S,6R)-2-(((2R,3R,4R,5S,6R)-3-amino-4,5-dihydroxy-6-(hydroxymethyl)tetrahydro-2H-pyran-2-yl)oxy)-6-(hydroxymethyl)tetrahydro-2H-pyran-3,4,5-triol (2-TreNH₂),
(2R,3R,4S,5S,6R)-2-(((2R,3R,4S,5S,6R)-4-amino-3,5-dihydroxy-6-(hydroxymethyl)tetrahydro-2H-pyran-2-yl)oxy)-6-(hydroxymethyl)tetrahydro-2H-pyran-3,4,5-triol (3-TreNH₂),
(2R,3R,4S,5S,6R)-2-(((2R,3R,4S,5S,6S)-5-amino-3,4-dihydroxy-6-(hydroxymethyl)tetrahydro-2H-pyran-2-yl)oxy)-6-(hydroxymethyl)tetrahydro-2H-pyran-3,4,5-triol (4-TreNH₂),
(2R,3S,4S,5R,6R)-2-(aminomethyl)-6-(((2R,3R,4S,5S,6R)-3,4,5-trihydroxy-6-(hydroxymethyl)tetrahydro-2H-pyran-2-yl)oxy)tetrahydro-2H-pyran-3,4,5-triol (6-TreNH₂),
(2R,3R,4S,5S,6R)-2-(((2R,3S,4R,5S,6R)-3-amino-4,5-dihydroxy-6-(hydroxymethyl)tetrahydro-2H-pyran-2-yl)oxy)-6-(hydroxymethyl)tetrahydro-2H-pyran-3,4,5-triol (2-epi-TreNH₂),
(2R,3R,4S,5S,6R)-2-(((2R,3R,4R,5S,6R)-4-amino-3,5-dihydroxy-6-(hydroxymethyl)tetrahydro-2H-pyran-2-yl)oxy)-6-(hydroxymethyl)tetrahydro-2H-pyran-3,4,5-triol (3-epi-TreNH₂),
(2R,3R,4S,5S,6R)-2-(((2R,3R,4S,5R,6S)-5-amino-3,4-dihydroxy-6-(hydroxymethyl)tetrahydro-2H-pyran-2-yl)oxy)-6-(hydroxymethyl)tetrahydro-2H-pyran-3,4,5-triol (4-epi-TreNH₂),
(2R,3R,4S,5R,6R)-2-(hydroxymethyl)-6-(((2R,3R,4S,5S,6R)-3,4,5-trihydroxy-6-(hydroxymethyl)tetrahydro-2H-pyran-2-yl)oxy) piperidine-3,4,5-triol (5-TreNH), or
a combination thereof.

15. The composition of claim 13 wherein the mycobacterial antibiotic is bedaquiline, ethambutol, isoniazid, pyrazinamide, rifampicin, streptomycin, ciprofloxacin, moxifloxacin, p-aminosalicylic acid, or a combination thereof.
